SEQUENCE LISTING (8 sequences)

SEQ ID NO: 1 - protein, 8 aa, Artificial Sequence
  Description of Artificial Sequence Synthetic peptide
DYKDDDDK

SEQ ID NO: 2 - protein, 23 aa, Artificial Sequence
  Description of Artificial Sequence Synthetic peptide
KRITVEEALAHPYLEQYYDPTDE

SEQ ID NO: 3 - protein, 6 aa, Artificial Sequence
  Description of Artificial Sequence 6xHis tag
HHHHHH

SEQ ID NO: 4 - protein, 10 aa, Artificial Sequence
  Description of Artificial Sequence Synthetic peptide
EQKLISEEDL

SEQ ID NO: 5 - protein, 9 aa, Artificial Sequence
  Description of Artificial Sequence Synthetic peptide
YPYDVPDYA

SEQ ID NO: 6 - protein, 11 aa, Artificial Sequence
  Description of Artificial Sequence Synthetic peptide
YTDIEMNRLGK

SEQ ID NO: 7 - protein, 11 aa, Artificial Sequence
  Description of Artificial Sequence Synthetic peptide
QPELAPEDPED

SEQ ID NO: 8 - protein, 9 aa, Artificial Sequence
  Description of Artificial Sequence Synthetic peptide
CDYKDDDDK

(19) United States
(12) Patent Application Publication
(10) Pub. No.: US 2019/0145971 A1
(43) Pub. Date: May 16, 2019

(54) Lateral Flow Analyte Detection

(71) Applicant: TGR BioSciences Pty Ltd., Thebarton (AU)

(72) Inventors: Antony James Sheehan, Seaview Downs (AU); Michael Francis Crouch, Hackney (AU)

(21) Appl. No.: 16/017,815

(22) Filed: Jun. 25, 2018

Related U.S. Application Data
(63) Continuation of application No. 15/222,376, filed on Jul. 28, 2016, now abandoned.
(60) Provisional application No. 62/355,133, filed on Jun. 27, 2016.

(51) Int. Cl. G01N 33/558

(52) U.S. Cl. CPC G01N 33/558

(57) ABSTRACT

The present disclosure relates to lateral flow methods and apparatuses for detecting one or more analytes. Certain embodiments provide a lateral flow device, kit and method of using the same, comprising: a flow path defined by a permeable sub-assembly of the lateral flow device, a release zone comprising a plurality of peptide-tagged agents, and a detection zone comprising a plurality of anti-peptide agents present in the flow path at a ratio of at least 100:1, on a weight:weight basis, relative to the plurality of peptide-tagged agents.

### CROSS-REFERENCE TO RELATED APPLICATIONS

This application is a continuation of U.S. patent application Ser. No. 15/222,376, filed Jul. 28, 2016, and which further claims the benefit of priority from U.S. Provisional Application No. 62/355,133, filed Jun. 27, 2016. The foregoing related applications, in their entirety, are incorporated herein by reference.

### SEQUENCE LISTING

The instant application contains a Sequence Listing which has been submitted electronically in ASCII format and is hereby incorporated by reference in its entirety. Said ASCII copy, created on Jul. 21, 2016, is named 013018-0009-999_SL.txt and is 2,160 bytes in size.

### FIELD OF THE INVENTION

The present disclosure relates to methods and apparatuses for detecting one or more analytes in a sample. In particular, this specification provides improved embodiments of lateral flow analyte detection methods and apparatuses.

### BACKGROUND OF THE INVENTION

Lateral flow assays combine various reagents and process steps in one container, thus providing relatively convenient and rapid detection of the presence, or absence, of a variety of analytes in liquid samples, including antigens such as hormones, e.g., human chorionic gonadotrophin (hCG) for pregnancy testing, antibodies such as antibodies to infectious agents, and haptens such as drugs of abuse.

Conventional lateral flow assays provide a permeable test strip configured to (a) expose an analyte-containing sample to a detectable agent which may bind with the analyte; and (b) passively transport (e.g., by capillary action) the analyte bound with the detectable agent to a detection zone of the test strip, where it is immobilized to the test strip in a matter of minutes. The analyte bound with the detectable agent may be immobilized, for example, with an immobilization agent present on the test strip. To facilitate passive transport, the test strip typically comprises one or more permeable materials in fluid communication.

A conventional lateral flow assay may be more expensive than a comparable plate assay employing the same type of immobilization agent because, inter alia, a sample may flow through the lateral flow test strip under the influence of capillary action in a matter of minutes. By way of comparison, a traditional plate assay may be configured to incubate the sample in the presence of an immobilization agent for a much longer period of time typically between 30 minutes and 2 hours. To compensate for reduced contact time, lateral flow assays are often configured with a higher concentration of immobilization agent and/or detectable agent, relative to comparable plate assays, in order to ensure that sufficient immobilization agent may be present to capture at least a detectable portion of the analyte, particularly when the analyte is expected to be present at low concentration in the sample. When the immobilization agent is expensive, the cost difference can become considerable (for example, an assay quantity of a monoclonal antibody immobilization agent may cost significantly more).

The need for a high concentration of detectable agent in a lateral flow assay may also impact test sensitivity, particularly if excess unbound detectable agent is difficult to wash from the test strip. Such excess unbound detectable agent may produce significant background noise that interferes with the detection signal.

In addition, fixing the immobilization agent and the other required assay components in a single combination on the test strip proscribes mixing and matching different assay component combinations, resulting in reduced flexibility and potential waste of assay components, for example if detection of a different type of analyte is required.

Accordingly, there is a need for lateral flow assays which are more flexible, more sensitive, and/or less expensive.

Reference to any prior art in this specification is not, and should not be taken as, an acknowledgment or any form of suggestion that this prior art forms part of the common general knowledge in any country.

### BRIEF SUMMARY OF THE INVENTION

Certain embodiments may provide an assay platform for detecting an analyte. In certain embodiments, the assay platform may comprise, for example, a permeable zone that may be configured, for example, to transport at least a portion of a sample that may comprise the analyte, and optionally a further permeable zone may be configured, for example, to receive at least a portion of the sample. In certain embodiments, the permeable zone may comprise, for example, a plurality of peptide-tagged agents. In certain embodiments, the plurality of peptide-tagged agents may be capable of forming an analyte complex that may comprise at least one of the plurality of peptide-tagged agents and the analyte. In certain embodiments, the further permeable zone may be configured to receive the portion of the sample from the permeable zone. In certain embodiments, the further permeable zone may comprise, for example, a plurality of anti-peptide agents present, for example, at a ratio of at least 100:1, for example at least 125:1, 200:1, or 300:1, on a weight:weight basis, relative to the plurality of peptide-tagged agents. In certain embodiments, the plurality of anti-peptide agents may be configured, for example, to immobilize the at least one of the plurality of peptide-tagged agents. In certain embodiments, the plurality of anti-peptide agents may be configured, for example, to immobilize the analyte complex. In certain embodiments, the plurality of anti-peptide agents may be bound to at least a portion of the further permeable zone. In certain embodiments, at least one of the at least one of the plurality of peptide-tagged agents and at least one of the plurality of anti-peptide agents may be binding partners.

In certain embodiments, the transporting may comprise, for example, passive transport, capillary action, wetting, wicking, or a combination of two or more thereof.

In certain embodiments, the at least one of the plurality of peptide-tagged agents may be bound to at least a portion of the further permeable zone. In certain embodiments, at least one of the at least one of the plurality of peptide-tagged agents may comprise, for example, a plurality of covalently conjugated peptide tags. In certain further embodiments, most or all of the plurality of covalently conjugated peptide tags present on the at least one of the plurality of peptide-tagged agents may comprise a single type of peptide tag. In certain further embodiments, most or all of the plurality of covalently conjugated peptide tags present on the at least one of the plurality of peptide-tagged agents may comprise two, three, or four types of peptide tags. In certain embodiments, most or all of the plurality of peptide-tagged agents may comprise a single type of peptide tag covalently conjugated thereon. In certain embodiments, most or all of the plurality of peptide-tagged agents may comprise two, three, or four types of peptide tags covalently conjugated thereon. In certain embodiments, most, for example a preponderance, or substantially all (in certain further embodiments greater than 90%, greater than 95% or greater than 98% of a certain type of bound peptide tags) of the one type of the plurality of peptide tags, that are bound, is covalently conjugated to only one type of the plurality of peptide-tagged agents.

In certain embodiments, at least one of the at least one of the plurality of peptide-tagged agents may comprise, for example, a capture agent capable of binding with the analyte, for example a monoclonal antibody, an affimer, or an aptamer capable of binding with the analyte. In certain further embodiments, each of the at least one of the plurality of peptide-tagged agents may comprise a single capture agent. In certain embodiments, the plurality of peptide-tagged agents may comprise a single type of capture agent. In certain embodiments, the plurality of peptide-tagged agents may comprise two, three, or four types of capture agents (and in some embodiments two, three or four types of peptide tags, and some embodiments just a single type of peptide tag per type of peptide tag).

In certain embodiments, at least one of the covalently conjugated peptide tags may be capable of binding with at least one of the plurality of anti-peptide agents. In certain further embodiments, the at least one of the plurality of anti-peptide agents may be a single anti-peptide agent. In certain embodiments, the plurality of anti-peptide agents may comprise a single type of anti-peptide agent. In certain embodiments, the plurality of anti-peptide agents may comprise two, three, or four types of anti-peptide agents.

In certain embodiments, the permeable zone may further comprise, for example, a plurality of detectable agents, and optionally the analyte complex may further comprise, for example, at least one of the plurality of detectable agents, for example one of the plurality of detectable agents. In certain embodiments, the plurality of detectable agents may comprise a single type of detectable agent. In certain embodiments, the plurality of detectable agents may comprise two, three, or four types of detectable agents. In certain embodiments, the plurality of anti-peptide agents may be present at a ratio of at least 8:1, on a weight:weight basis, relative to the plurality of detectable agents.

In certain embodiments, the assay platform or components or sub-assemblies thereof may be stored at a predetermined temperature, for example, at a temperature of 4° C., room temperature, less than 10° C., less than 20° C., at a temperature of less than 30° C. In certain embodiments, the assay platform or components or sub-assemblies thereof may be stored prior to use and/or after use at a predetermined temperature, for example, at a temperature of 4° C., room temperature, less than 10° C., less than 20° C., or at a temperature of less than 30° C. In certain embodiments, the assay platform may have a shelf-life of at least two months at the pre-determined temperature, for example a shelf-life of at least 2 months, 6 months, or at least 1 year. In certain embodiments, the assay platform may have a shelf-life of at least two months at a pre-determined temperature of storage prior to use of less than 20° C., for example a shelf-life of at least 2 months at a pre-determined temperature of storage prior to use of less than 20° C., 6 months at a pre-determined temperature of storage prior to use of less than 20° C., or at least 1 year at a pre-determined temperature of storage prior to use of less than 30° C.

In certain embodiments, the assay platform or components or sub-assemblies thereof, for example the plurality of peptide-tagged agents and/or the plurality of anti-peptide agents, may be stored under low light conditions, such as in a sealed container. In certain embodiments, the assay platform or components or sub-assemblies thereof, for example the plurality of peptide-tagged agents and/or the plurality of anti-peptide agents, may be stored prior to use and/or after use under low light conditions, such as in a sealed container.

Certain embodiments may provide, for example, an assay platform for detecting a pre-determined analyte, comprising: i) a permeable zone configured to transport a sample comprising the pre-determined analyte, said permeable zone comprising a plurality of peptide-tagged agents, said plurality of peptide-tagged agents capable of forming an analyte complex comprising at least one of the plurality of peptide-tagged agents and the pre-determined analyte; and ii) a further permeable zone configured to receive at least a portion of the sample from the permeable zone, the further permeable zone comprising: a plurality of anti-peptide agents present at a ratio of at least 100:1, on a weight:weight basis, relative to the plurality of peptide-tagged agents, said plurality of anti-peptide agents configured to immobilize the at least one of the plurality of peptide-tagged agents. In certain embodiments, the plurality of anti-peptide agents may be configured, for example, to immobilize the analyte complex.

Certain embodiments may provide a lateral flow device. In certain embodiments, the lateral flow device may comprise, for example, a flow path that may be defined by, for example, at least a portion of a permeable sub-assembly of the lateral flow device. In certain embodiments, the lateral flow device may further comprise, for example, a release zone that may comprise, for example, a plurality of peptide-tagged agents. In certain embodiments, the lateral flow device may further comprise, for example, a detection zone that may comprise, for example, a plurality of anti-peptide agents that may be present, for example, in the flow path at a ratio of at least 100:1, on a weight:weight basis, relative to the plurality of peptide-tagged agents. In certain embodiments, the release zone may be configured, for example, to release at least a portion of said plurality of peptide-tagged agents into the flow path. In certain embodiments, at least one of said plurality of anti-peptide agents may be a binding partner with at least one of the at least a portion of said plurality of peptide-tagged agents configured to be released.

In certain embodiments, at least a portion of the flow path may be characterized by passive transport, capillary action, wetting, wicking, or a combination of two or more thereof. In certain embodiments, the plurality of peptide-tagged agents may be bound, releasably bound, or unbound to at least a portion of the permeable sub-assembly. In certain embodiments, at least a portion (for example, most, a preponderance, or substantially all (in certain further embodiments greater than 90%, greater than 95% or greater than 98% on a weight:weight basis)) of the plurality of peptide-tagged agents may migrate, dissolve, disconnect, and/or diffuse from at least a portion of the permeable sub-assembly.

In certain embodiments, the plurality of anti-peptide agents may be bound to at least a portion of the permeable sub-assembly. In certain embodiments, the at least a portion of the at least a portion of said plurality of peptide-tagged agents configured to be released may be bound to at least a further portion of the permeable sub-assembly. In certain embodiments, said plurality of anti-peptide agents may be configured, for example, to immobilize at least a portion of the at least a portion of said plurality of peptide-tagged agents configured to be released. In certain further embodiments, at least one of the at least a portion of said plurality of peptide-tagged agents configured to be released may be capable of binding (and may, for example, be bound) with an analyte prior to being immobilized by said plurality of anti-peptide agents. In certain embodiments, at least one of the at least a portion of the plurality of peptide-tagged agents may comprise, for example, a plurality of covalently conjugated peptide tags, wherein at least one of said covalently conjugated peptide tags may be capable of binding (and may, for example, be bound) with at least one of the plurality of anti-peptide agents. In certain further embodiments, the at least one of the plurality of anti-peptide agents may be one of the plurality of anti-peptide agents. In certain embodiments, the detection zone may be positioned in the flow path downstream of the release zone.

In certain embodiments, the release zone may further comprise, for example, a plurality of detectable agents, and the analyte complex may further comprise, for example, at least one of the plurality of detectable agents. In certain further embodiments, the plurality of detectable agents may be bound, releasably bound, or unbound to the permeable sub-assembly. In certain further embodiments, the release zone may be configured to further release at least a portion of said plurality of detectable agents into the flow path. In certain further embodiments, at least a portion of the plurality of detectable agents may be capable of migrating, dissolving, disconnecting, or diffusing (and may, for example, migrate, dissolve, disconnect, or diffuse) from at least a portion of the permeable sub-assembly.

Certain embodiments may provide, for example, a lateral flow device, comprising: (i) a flow path defined by a permeable sub-assembly of the lateral flow device; (ii) a release zone comprising a plurality of peptide-tagged agents, said release zone configured to release at least a portion of said plurality of peptide-tagged agents into the flow path; and (iii) a detection zone comprising a plurality of anti-peptide agents present in the flow path at a ratio of at least 100:1, on a weight:weight basis, relative to the plurality of peptide-tagged agents, wherein at least one of said plurality of anti-peptide agents is a binding partner with at least one of the released at least a portion of said plurality of peptide-tagged agents.

Certain embodiments may provide a lateral flow device. In certain embodiments, the lateral flow device may be configured to detect at least two analytes, for example at least three analytes, at least four analytes, at least five analytes, at least six analytes, at least seven analytes, at least eight analytes, at least nine analytes, or configured to detect at least ten analytes. In certain embodiments, the lateral flow device may be configured to detect up to ten analytes. In certain embodiments, the lateral flow device may comprise, for example, a flow path that may be defined by, for example, at least a portion of a permeable sub-assembly of the lateral flow device. In certain embodiments, the lateral flow device may further comprise, for example, a release zone that may comprise, for example, a plurality of first peptide-tagged agents. In certain embodiments, the lateral flow device may further comprise, for example, a detection zone that may comprise, for example, a plurality of first anti-peptide agents that may be present, for example, in the flow path at a ratio of at least 100:1, on a weight:weight basis, relative to the plurality of first peptide-tagged agents. In certain embodiments, the release zone may be configured, for example, to release at least a portion of said plurality of first peptide-tagged agents into the flow path. In certain embodiments, the at least one of said plurality of first anti-peptide agents may be configured to immobilize at least one of said plurality of first peptide-tagged agents configured to be released. In certain embodiments, the release zone may also comprise, for example, a plurality of second peptide-tagged agents. In certain embodiments, the detection zone may also comprise, for example, a plurality of second anti-peptide agents that may be configured to immobilize at least one of the at least a portion of said plurality of second peptide-tagged agents configured to be released.

In certain embodiments, the plurality of first peptide-tagged agents may comprise, for example, a first type of peptide tag, and the plurality of second peptide-tagged agents may comprise, for example, a second type of peptide tag. In certain embodiments, the first type of peptide tag and the second type of peptide tag may be different types of peptide tags and/or sequences of peptides. In certain embodiments, at least one of the plurality of first peptide-tagged agents may comprise, for example, a plurality of covalently conjugated first peptide tags. In certain further embodiments, most or all of the plurality of covalently conjugated first peptide tags present on the at least one of the plurality of first peptide-tagged agents may comprise a first type of peptide tag. In certain embodiments, most or all of the plurality of first peptide-tagged agents may comprise the first type of peptide tag covalently conjugated thereon. In certain embodiments, at least one of the plurality of second peptide-tagged agents may comprise, for example, a plurality of covalently conjugated second peptide tags. In certain further embodiments, most or all of the plurality of covalently conjugated second peptide tags present on the at least one of the plurality of second peptide-tagged agents may comprise a second type of peptide tag. In certain embodiments, most or all of the plurality of second peptide-tagged agents may comprise the second type of peptide tag covalently conjugated thereon.

In certain embodiments, at least one of the plurality of first peptide-tagged agents may comprise, for example, a first capture agent, for example a first monoclonal antibody, a first affimer, or a first aptamer, capable of binding with a first analyte. In certain further embodiments, each of the at least one of the plurality of first peptide-tagged agents may comprise a single first capture agent. In certain embodiments, the plurality of first peptide-tagged agents may comprise a single type of first capture agent. In certain embodiments, at least one of the plurality of second peptide-tagged agents may comprise, for example, a second capture agent, for example a second monoclonal antibody, a second affimer, or a second aptamer, capable of binding with a second analyte. In certain further embodiments, each of the at least one of the plurality of second peptide-tagged agents may comprise a single second capture agent. In certain embodiments, the plurality of second peptide-tagged agents may comprise a single type of second capture agent.

In certain embodiments, the plurality of first anti-peptide agents may comprise a first type of anti-peptide agent, and the plurality of second anti-peptide agents may comprise a second type of anti-peptide agent. In certain embodiments, the first type of anti-peptide agent and the second type of anti-peptide agent may be different types of anti-peptide agents. In certain further embodiments, the first type of anti-peptide agent may be capable of binding with the first type of peptide tag, and the second type of anti-peptide agent may be capable of binding with the second type of peptide tag. In certain further embodiments, the first type of anti-peptide agent may be a specific binding partner with the first type of peptide tag, and the second type of anti-peptide agent may be a specific binding partner with the second type of peptide tag.

In certain embodiments, the plurality of first anti-peptide agents may be interspersed with the plurality of second anti-peptide agents. In certain embodiments, at least a portion of the plurality of first anti-peptide agents may be segregated from at least a portion of the plurality of second anti-peptide agents. In certain embodiments, at least a portion of the plurality of first anti-peptide agents may be downstream from at least a portion of the plurality of second anti-peptide agents, relative to the direction of flow of the flow path. In certain embodiments, at least a portion of the plurality of first anti-peptide agents and at least a portion of the plurality of second anti-peptide agents may be segregated from one another and positioned side by side, relative to a direction of flow defined by the flow path.

Certain embodiments may provide, for example, a lateral flow device, comprising: (i) a flow path defined by a permeable sub-assembly of the lateral flow device; (ii) a release zone comprising: (a) a plurality of first peptide-tagged agents, said release zone configured to release at least a portion of said plurality of first peptide-tagged agents into the flow path; and (b) a plurality of second peptide-tagged agents, said release zone configured to release at least a portion of said plurality of second peptide-tagged agents into the flow path; and (iii) a detection zone comprising: (a) a plurality of first anti-peptide agents present in the flow path at a ratio of at least 100:1, on a weight:weight basis, relative to the plurality of first peptide-tagged agents, wherein at least one of said plurality of first anti-peptide agents is a binding partner with at least one of the configured to be released at least a portion of said plurality of first peptide-tagged agents; and (b) a plurality of second anti-peptide agents present in the flow path at a ratio of at least 100:1, on a weight:weight basis, relative to the plurality of second peptide-tagged agents, wherein at least one of said plurality of second anti-peptide agents is a binding partner with at least one of the configured to be released at least a portion of said plurality of second peptide-tagged agents.

Certain embodiments may provide an assembly. In certain embodiments, the assembly may comprise, for example, a permeable member that may have a plurality of peptide-tagged agents deposited on at least a portion thereof. In certain embodiments, the assembly may further comprise, for example, a further permeable member that may be in fluid communication with the permeable member. In certain embodiments, the plurality of peptide-tagged agents may be capable of forming an analyte complex. In certain embodiments, the analyte complex may comprise, for example, at least one of the plurality of peptide-tagged agents and an analyte. In certain embodiments, the further permeable member may have a plurality of anti-peptide agents bound to at least a portion thereof. In certain embodiments, the plurality of anti-peptide agents may be present, for example, at a ratio of at least 100:1, on a weight:weight basis, relative to the plurality of peptide-tagged agents. Furthermore, in certain embodiments the plurality of anti-peptide agents may be capable of immobilizing at least one of said at least one of the plurality of peptide-tagged agents.

In certain embodiments, fluid communication of the plurality of peptide-tagged agents and/or the analyte between the permeable member and the further permeable member may comprise, for example, passive transport, capillary action, wetting, wicking, or a combination of two or more thereof. In certain embodiments, the plurality of peptide-tagged agents may be bound, releasably bound, or unbound to at least a portion of the permeable member. In certain embodiments, at least a portion of the plurality of peptide-tagged agents may migrate, dissolve, disconnect, and/or diffuse from at least a portion of the permeable member. In certain embodiments, at least one of the at least one of the plurality of peptide-tagged agents and at least one of the plurality of peptide-tagged agents may be binding partners. In certain embodiments, at least one of the at least one of the plurality of peptide-tagged agents may comprise, for example, a plurality of covalently conjugated peptide tags, wherein at least one of said covalently conjugated peptide tags may be capable of binding with at least one of the plurality of anti-peptide agents. In certain further embodiments, the at least one of the plurality of anti-peptide agents may be a single anti-peptide agent. In certain embodiments, the permeable member may be configured, for example, to fluidly communicate the analyte complex to the further permeable member. In certain embodiments, the permeable member may further comprise, for example, a plurality of detectable agents, and optionally the analyte complex may further comprise, for example, at least one of the plurality of detectable agents. In certain further embodiments, the plurality of detectable agents may be bound, releasably bound, or unbound to the permeable member. In certain further embodiments, at least a portion of the plurality of detectable agents can migrate, dissolve, disconnect, and/or diffuse from at least a portion of the permeable member.

Certain embodiments may provide, for example, an assembly, comprising: (i) a permeable member having a plurality of peptide-tagged agents deposited on at least a portion thereof, said plurality of peptide-tagged agents capable of forming an analyte complex, said analyte complex comprising at least one of the plurality of peptide-tagged agents and a pre-determined analyte; and (ii) a further permeable member in fluid communication with the permeable member, said further permeable member having a plurality of anti-peptide agents bound to at least a portion thereof, said plurality of anti-peptide agents present at a ratio of at least 100:1, on a weight:weight basis, relative to the plurality of peptide-tagged agents, said plurality of anti-peptide agents capable of immobilizing at least one of said at least one of the plurality of peptide-tagged agents.

Certain embodiments may utilize the assembly in a method to detect an analyte present at a low concentration, for example at a concentration of less than 1 mIU/mL in a sample, or at a concentration of less than 1 mM in a sample, or at a concentration of less than 10 ng/mL. In certain embodiments, the analyte concentration may be in the range of 0.001-1 mM, for example an analyte concentration in the range of 0.01-1 mM, 0.1-1 mM, 0.25-1 mM, or an analyte concentration in the range of 0.25-0.75 mM. In certain embodiments, the analyte concentration may be in the range of 0.001-10 ng/mL, for example an analyte concentration in the range of 0.01-10 ng/mL, 0.1-10 ng/mL, 1-7.5 ng/mL, or an analyte concentration in the range of 2.5-7.5 ng/mL. In certain embodiments, the method may comprise depositing a sample onto the apparatus. In certain further embodiments, the method may comprise detecting the target analyte by measuring a detection signal having a signal-to-noise ratio of at least 1.67. In certain embodiments, the signal to noise ratio may be defined as the ratio of the detection signal to a detection signal obtained if the apparatus is configured and/or used in a similar manner, for example if the apparatus comprises the same or a similar amount of the plurality of peptide-tagged agent, the the same or a similar amount of the plurality of anti-peptide agent, and/or the the same or a similar amount of the plurality of detectable agent, but where there is no analyte present.

Certain embodiments may provide an assembly. In certain embodiments, the assembly may comprise, for example, a permeable member that may have a plurality of first peptide-tagged agents deposited on at least a portion thereof. In certain embodiments, the assembly may further comprise, for example, a further permeable member that may be in fluid communication with the permeable member. In certain embodiments, the plurality of first peptide-tagged agents may be capable of forming a first analyte complex. In certain embodiments, the first analyte complex may comprise, for example, at least one of the plurality of first peptide-tagged agents and a first analyte. In certain embodiments, the further permeable member may have a plurality of first anti-peptide agents bound to at least a portion thereof. In certain embodiments, the plurality of first anti-peptide agents may be present, for example, at a ratio of at least 100:1, on a weight:weight basis, relative to the plurality of first peptide-tagged agents. In certain further embodiments, the plurality of first anti-peptide agents may be capable of immobilizing at least one of said at least one of the plurality of first peptide-tagged agents. In certain embodiments, the permeable member may also have, for example, a plurality of second peptide-tagged agents deposited on at least a portion thereof. In certain embodiments, the plurality of second peptide-tagged agents may be capable of forming a second analyte complex. In certain embodiments, the second analyte complex may comprise, for example, at least one of the plurality of second peptide-tagged agents and a second analyte. In certain embodiments, the further permeable member may also have, for example, a plurality of second anti-peptide agents bound to at least a portion thereof. In certain embodiments, the plurality of second anti-peptide agents may be capable of immobilizing at least one of said at least one of the plurality of second peptide-tagged agents.

In certain embodiments, the plurality of first peptide-tagged agents and the plurality of second peptide-tagged agents may be interspersed on the permeable member. In certain embodiments, the plurality of first peptide-tagged agents and the plurality of second peptide-tagged agents, or portions thereof, may be segregated from one another on the permeable member.

In certain embodiments, the plurality of first peptide-tagged agents and the plurality of second peptide-tagged agents may comprise two or more different types of peptide-tagged agents, for example two different types of peptide-tagged agents. In certain embodiments, the plurality of first peptide-tagged agents and the plurality of second peptide-tagged agents may comprise two or more different types of capture agents, for example two different types of capture agents. In certain embodiments, the plurality of first peptide-tagged agents and the plurality of second peptide-tagged agents may comprise two or more different types of covalently conjugated peptide tags, for example two different types of peptide-tags.

In certain embodiments, the plurality of first anti-peptide agents and the plurality of second anti-peptide agents may comprise two or more different types of anti-peptide agents, for example two different types of anti-peptide agents. In certain embodiments, the plurality of first anti-peptide agents and the plurality of second anti-peptide agents may have one or more type of anti-peptide agents in common, for example one type of anti-peptide agent in common.

In certain embodiments, the plurality of first anti-peptide agents and the plurality of second anti-peptide agents may be interspersed on the permeable member. In certain embodiments, the plurality of first anti-peptide agents and the plurality of second anti-peptide agents, or portions thereof, may be segregated from one another on the permeable member.

In certain embodiments, at least a portion of the plurality of first anti-peptide agents may be bound in a first sub-zone of the detection zone. In certain embodiments, at least a portion of the plurality of second anti-peptide agents may be bound in a second sub-zone of the detection zone. In certain embodiments, the first sub-zone and the second sub-zone may be overlapping. In certain embodiments, the first sub-zone and the second sub-zone may be disjoint. In certain embodiments, a portion of the sample, or a portion of the portion of the sample, may migrate through the first sub-zone before said portion migrates through the second sub-zone.

Certain embodiments may provide, for example, an assembly, comprising: (i) a permeable member having: (a) a plurality of first peptide-tagged agents deposited on at least a portion thereof, said plurality of first peptide-tagged agents capable of forming a first analyte complex, said first analyte complex comprising at least one of the plurality of first peptide-tagged agents and a first pre-determined analyte; and (b) a plurality of second peptide-tagged agents deposited on at least a portion thereof, said plurality of second peptide-tagged agents capable of forming a second analyte complex, said second analyte complex comprising at least one of the plurality of second peptide-tagged agents and a second pre-determined analyte; and (ii) a further permeable member in fluid communication with the permeable member, said further permeable member having: (a) a plurality of first anti-peptide agents bound to at least a portion thereof, said plurality of first anti-peptide agents present at a ratio of at least 100:1, on a weight:weight basis, relative to the plurality of first peptide-tagged agents, said plurality of first anti-peptide agents capable of immobilizing at least one of the said at least one of the plurality of first peptide-tagged agents; and (b) a plurality of second anti-peptide agents bound to at least a portion thereof, said plurality of second anti-peptide agents present at a ratio of at least 100:1, on a weight:weight basis, relative to the plurality of second peptide-tagged agents, said plurality of second anti-peptide agents capable of immobilizing at least one of the said at least one of the plurality of second peptide-tagged agents.

Certain embodiments may provide a permeable assembly. In certain embodiments, the permeable assembly may comprise, for example, a flow path defined by, for example, the permeable assembly. In certain embodiments, the permeable assembly may further comprise, for example, a sample zone. In certain embodiments, the permeable assembly may further comprise, for example, a detection zone. In certain embodiments, the sample zone may comprise, for example, a plurality of analyte complexes. In certain further embodiments, said plurality of analyte complexes may comprise, for example, a plurality of peptide-tagged agents. In certain embodiments, the detection zone may comprise, for example, a plurality of anti-peptide agents present, for example, in the flow path at a ratio of at least 100:1, on a weight:weight basis, relative to the plurality of peptide-tagged agents. In certain embodiments, the plurality of anti-peptide agents may be capable of immobilizing at least a portion of the plurality of peptide-tagged agents.

In certain embodiments, the sample zone may be configured, for example, to release at least a portion of said plurality of analyte complexes into the flow path. In certain embodiments, the plurality of anti-peptide agents may be positioned in the flow path downstream of the sample zone. In certain embodiments, the sample zone and the detection zone may be in spatial relation to one another, for example abutting, adjacent, and/or collinear to one another. In certain embodiments, the detection zone may be housed, for example, in a casing. In certain further embodiments, the casing may define, for example, a viewing hole, a window, and/or a transparent section positioned proximate the detection zone. In certain further embodiments, the sample zone may be housed, for example, in the casing. In certain further embodiments, the sample zone may be configured, for example, to receive the plurality of analytes through a sample inlet defined by, for example, the casing.

Certain embodiments may provide, for example, a permeable assembly, comprising: (i) a flow path defined by the permeable assembly; (ii) a sample zone, comprising: a plurality of analyte complexes, said plurality of analyte complexes comprising a plurality of peptide-tagged agents; and (iii) a detection zone, comprising: a plurality of anti-peptide agents present in the flow path at a ratio of at least 100:1, on a weight:weight basis, relative to the plurality of peptide-tagged agents, said plurality of anti-peptide agents capable of immobilizing at least a portion of the plurality of peptide-tagged agents.

Certain embodiments may provide an assembly. In certain embodiments, the assembly may comprise, for example, a permeable member having a plurality of analyte complexes deposited on at least a portion thereof. In certain embodiments, the assembly may further comprise, for example, a further permeable member that may be configured, for example, to receive at least a portion of the analyte complexes from the permeable member. In certain embodiments, the plurality of analyte complexes may comprise, for example, a plurality of peptide-tagged agents. In certain embodiments, the further permeable member may have a plurality of anti-peptide agents bound to at least a portion thereof. In certain embodiments, the plurality of anti-peptide agents may be present, for example, at a ratio of at least 100:1, on a weight:weight basis, relative to the plurality of peptide-tagged agents. Furthermore, in certain embodiments the plurality of anti-peptide agents may be configured, for example, to immobilize said at least one of the plurality of peptide-tagged agents to the permeable assembly.

In certain embodiments, the permeable member and the further permeable member may be in spatial relation to one another, for example abutting, adjacent, and/or collinear to one another. In certain embodiments, the permeable member may comprise, for example, an absorbent pad. In certain embodiments, the permeable member may be housed, for example, in a casing. In certain further embodiments, the permeable member may be configured, for example, to receive the plurality of analytes through a sample inlet defined by, for example, the casing. In certain embodiments, the further permeable member may comprise, for example, at least one porous membrane. In certain embodiments, the further permeable member may be housed, for example, in the casing. In certain further embodiments, the casing defines, for example, a viewing hole, window, and/or a transparent section positioned proximate the further permeable member. In certain embodiments, the assembly may further may comprise, for example, an absorbent member positioned in the flow path downstream of the further permeable member.

Certain embodiments may provide, for example, an assembly, comprising: (i) a permeable member having a plurality of analyte complexes deposited on at least a portion thereof, said permeable member configured to transport at least a portion of the analyte complexes, said plurality of analyte complexes comprising a plurality of peptide-tagged agents; and (ii) a further permeable member configured to receive at least the portion of the analyte complexes from the permeable member, said further permeable member having a plurality of anti-peptide agents bound to at least a portion thereof, said plurality of anti-peptide agents: (a) present at a ratio of at least 100:1, on a weight:weight basis, relative to the plurality of peptide-tagged agents; and (b) configured to immobilize said at least one of the plurality of peptide-tagged agents to the permeable assembly.

Certain embodiments may provide a lateral flow device for detecting an analyte. In certain embodiments, the lateral flow device may comprise, for example, a flow path defined by, for example, a permeable sub-assembly of the lateral flow device. In certain embodiments, the lateral flow device may further comprise, for example, a sample zone that may be configured, for example, to receive the analyte and to release the analyte into the flow path. In certain embodiments, the lateral flow device may further comprise, for example, a release zone that may comprise a plurality of peptide-tagged agents. In certain embodiments, the lateral flow device may further comprise, for example, a detection zone that may comprise a plurality of anti-peptide agents present, for example, in the flow path at a ratio of at least 100:1, on a weight:weight basis, relative to the plurality of peptide-tagged agents. In certain embodiments, the release zone may be configured, for example, to release at least a portion of said plurality of peptide-tagged agents into the flow path. In certain embodiments, at least one of said plurality of anti-peptide agents may be a binding partner with at least one of the at least a portion of said plurality of peptide-tagged agents configured to be released.

In certain embodiments, the release zone may be positioned in the flow path downstream of the sample zone. In certain embodiments, the detection zone may be positioned in the flow path downstream of the release zone. In certain embodiments, the sample zone and the release zone may be in spatial relation to one another, for example abutting, adjacent, and/or collinear to one another. In certain embodiments, the release zone and the detection zone may be in spatial relation to one another, for example abutting, adjacent, and/or collinear to one another. In certain embodiments, the sample zone and the detection zone may not be in spatial relation to one another, for example not abutting, not adjacent, and not collinear to one another. In certain embodiments, the release zone and the detection zone may be housed, for example, in a casing. In certain further embodiments, the sample zone may be housed, for example, in the casing. In certain further embodiments, the sample zone may be configured, for example, to receive the analyte through a sample inlet defined by, for example, the casing. In certain embodiments, the lateral flow device may further comprise, for example, a plurality of detectable agents. In certain further embodiments, the plurality of detectable agents may be introduced to the permeable sub-assembly separately from the analyte. In certain further embodiments, the plurality of detectable agents may be bound to the permeable sub-assembly upstream of, downstream of, side by side with, or interspersed with said plurality of peptide-tagged agents.

Certain embodiments may provide, for example, a lateral flow device for detecting a pre-determined analyte, comprising: (i) a flow path defined by a permeable sub-assembly of the lateral flow device; (ii) a sample zone configured to receive the pre-determined analyte and to release the pre-determined analyte into the flow path; (iii) a release zone comprising a plurality of peptide-tagged agents, said release zone configured to release at least a portion of said plurality of peptide-tagged agents into the flow path; and (iv) a detection zone comprising a plurality of anti-peptide agents present in the flow path at a ratio of at least 100:1, on a weight:weight basis, relative to the plurality of peptide-tagged agents, wherein at least one of said plurality of anti-peptide agents is a binding partner with at least one of the released at least a portion of said plurality of peptide-tagged agents.

Certain embodiments may provide a lateral flow device. In certain embodiments, the lateral flow device may comprise, for example, a first permeable member that may be configured, for example, to receive a sample containing an analyte and, optionally, to transport said analyte. In certain embodiments, the lateral flow device may further comprise a second permeable member that may be configured, for example, to receive the analyte from the first member. In certain embodiments, the second permeable member may have a plurality of peptide-tagged agents deposited on at least a portion thereof. In certain embodiments, the plurality of peptide-tagged agents may be capable of forming an analyte complex. In certain embodiments, the analyte complex may comprise, for example, at least one of the plurality of peptide-tagged agents and the analyte. In certain embodiments, the lateral flow device may further comprise, for example, a third permeable member that may be configured, for example, to receive the analyte complex from the second permeable member. In certain embodiments, the third permeable member may have a plurality of anti-peptide agents bound to at least a portion thereof. In certain embodiments, the plurality of anti-peptide agents may be present, for example, at a ratio of at least 100:1, on a weight:weight basis, relative to the plurality of peptide-tagged agents. Furthermore, in certain embodiments, the plurality of anti-peptide agents may be configured, for example, to immobilize at least one of said at least one of the plurality of peptide-tagged agents.

In certain embodiments, the first permeable member and the second permeable member may be in spatial relation to one another, for example abutting, adjacent, and/or collinear to one another. In certain embodiments, the second permeable member and the third permeable member may be in spatial relation to one another, for example abutting, adjacent, and/or collinear to one another. In certain embodiments, the first permeable member and the third permeable member may not be in spatial relation to one another, for example not abutting, not adjacent, and not collinear to one another. In certain embodiments, second permeable member and the third permeable member may be housed, for example, in a casing. In certain further embodiments, the first permeable member may be housed, for example, in the casing. In certain further embodiments, the first permeable member may be configured, for example, to receive the sample through a sample inlet defined by, for example, the casing. In certain embodiments, the lateral flow device may further comprise, for example, a plurality of detectable agents. In certain further embodiments, the analyte complex may further comprise, for example, the analyte bound to one or more of the plurality of detectable agents, for example one of the plurality of detectable agents. In certain further embodiments, the plurality of detectable agents may be introduced to the lateral flow device separately from the sample. In certain further embodiments, the plurality of detectable agents may be bound to the second permeable member upstream of, downstream of, side by side with, or interspersed with said plurality of peptide-tagged agents. In certain embodiments, the lateral flow device may further comprise, for example, an absorbent member positioned in the flow path downstream of the first, second, and third permeable members. In certain further embodiments, the absorbency of the absorbent member may exceed the absorbency of the first permeable member, for example the quantity of a liquid which may be absorbed by the absorbent member may exceed that of the first permeable member by a factor of at least 2, for example at least 5, 10, 25, 50, or at least 100.

Certain embodiments may provide, for example, a lateral flow device, comprising: (i) a first permeable member configured to receive a sample containing a pre-determined analyte and to transport said pre-determined analyte; (ii) a second permeable member configured to receive the pre-determined analyte from the first member, said second permeable member having a plurality of peptide-tagged agents deposited on at least a portion thereof, said plurality of peptide-tagged agents capable of forming an analyte complex, said analyte complex comprising at least one of the plurality of peptide-tagged agents and the pre-determined analyte; and (iii) a third permeable member configured to receive the analyte complex from the second permeable member, said third permeable member having a plurality of anti-peptide agents bound to at least a portion thereof, said plurality of anti-peptide agents: (a) present at a ratio of at least 100:1, on a weight:weight basis, relative to the plurality of peptide-tagged agents; and (b) configured to immobilize at least one of the said at least one of the plurality of peptide-tagged agents.

Certain embodiments may provide a lateral flow device for detecting an analyte. In certain embodiments, the lateral flow device may comprise, for example, a flow path defined by, for example, a permeable sub-assembly of the lateral flow device. In certain embodiments, the lateral flow device may further comprise a solvent zone that may be configured, for example, to receive a solvent and, optionally, to communicate a portion of said solvent into the flow path. In certain embodiments, the lateral flow device may be configured, for example, to receive a sample in a sample zone of the lateral flow device and, optionally, to introduce at least a portion of said sample into the flow path. In certain embodiments, the lateral flow device may further comprise a release zone that may comprise a plurality of peptide-tagged agents. In certain embodiments, the lateral flow device may further comprise a detection zone that may comprise a plurality of anti-peptide agents present, for example, in the flow path at a ratio of at least 100:1, on a weight:weight basis, relative to the plurality of peptide-tagged agents. In certain embodiments, the at least a portion of said sample may contain the analyte. In certain embodiments, the release zone may be configured, for example, to release at least a portion of said plurality of peptide-tagged agents into the flow path. In certain embodiments, the plurality of anti-peptide agents may be configured, for example, to immobilize at least a fraction of the at least a portion of said plurality of peptide-tagged agents configured to be released. In certain embodiments, the sample zone may be positioned in the flow path downstream of the release zone.

In certain embodiments, the release zone may be positioned in the flow path downstream of the sample zone. In certain embodiments, the sample may be a solid, a gel, a dense sample, and/or a congealed sample. In certain embodiments, the sample may dissolve, mix, passively mix, and/or diffuse into the solvent. In certain embodiments, the analyte may migrate, dissolve, disconnect, and/or diffuse from the sample into the solvent. In certain embodiments, the release zone may be housed, for example, in a casing. In certain further embodiments, the sample zone may be housed, for example, in a casing. In certain further embodiments, the sample may be introduced to the lateral flow device through a sample inlet defined by, for example, the casing. In certain embodiments, the solvent zone may be configured, for example, to receive the solvent through a solvent inlet defined by, for example, the casing. In certain further embodiments, the sample inlet may be shaped to allow the sample to be spread, pipetted, and/or smeared onto the sample zone.

Certain embodiments may provide, for example, a lateral flow device for detecting a pre-determined analyte, comprising: (i) a flow path defined by a permeable sub-assembly of the lateral flow device; (ii) a solvent zone configured to receive a solvent and to communicate a portion of said solvent into the flow path; (iii) a sample zone configured to receive a sample and to introduce at least a portion of said sample into the flow path, said at least a portion of said sample containing the pre-determined analyte; (iv) a release zone comprising a plurality of peptide-tagged agents, said release zone configured to release at least a portion of said plurality of peptide-tagged agents into the flow path; and (v) a detection zone comprising a plurality of anti-peptide agents present in the flow path at a ratio of at least 100:1, on a weight:weight basis, relative to the plurality of peptide-tagged agents, said plurality of anti-peptide agents configured to immobilize at least a portion of the released at least a portion of said plurality of peptide-tagged agents.

Certain embodiments may provide a lateral flow device. In certain embodiments, the lateral flow device may comprise, for example, a first permeable member that may be configured, for example, to receive a solvent. In certain embodiments, the lateral flow device may further comprise, for example, a second permeable member that may be in fluid communication with the first permeable member. In certain embodiments, the lateral flow device may further comprise, for example, a third permeable member that may be in fluid communication with the first permeable member. In certain embodiments, the lateral flow device may further comprise, for example, a fourth permeable member that may be in fluid communication with at least the second and the third permeable members. In certain embodiments, the second permeable member may be configured, for example, to receive a sample containing an analyte. In certain embodiments, the third permeable member may have a plurality of peptide-tagged agents deposited thereon. In certain embodiments, the fourth permeable member may have a plurality of anti-peptide agents bound thereto at a ratio of at least 100:1, on a weight:weight basis, relative to the plurality of peptide-tagged agents.

Certain embodiments may provide, for example, a lateral flow device, comprising: (i) a first permeable member configured to receive a solvent; (ii) a second permeable member in fluid communication with the first permeable member, said second permeable member configured to receive a sample containing a pre-determined analyte; (iii) a third permeable member in fluid communication with the first permeable member, said third permeable member having a plurality of peptide-tagged agents deposited thereon; and (iv) a fourth permeable member in fluid communication with at least the second and the third permeable members, said fourth permeable member having a plurality of anti-peptide agents bound thereto at a ratio of at least 100:1, on a weight:weight basis, relative to the plurality of peptide-tagged agents.

Certain embodiments provide lateral flow methods, including, for example, methods of detection, methods of making lateral flow apparatuses, and methods of making lateral flow kits. Other types of lateral flow methods are contemplated herein.

Certain embodiments may provide a method. In certain embodiments, the method may comprise, for example, exposing a sample that may comprise an analyte to a plurality of peptide-tagged agents to form an analyte complex. In certain further embodiments, the method may comprise, for example, exposing the analyte complex to a plurality of detectable agents, to result in an analyte complex comprising at least one of the plurality of detectable agents. In certain embodiments, the method may comprise, for example, exposing a sample that may comprise an analyte to a plurality of detectable agents to form an analyte complex. In certain further embodiments, the method may comprise, for example, exposing the analyte complex to a plurality of peptide-tagged agents, to result in an analyte complex comprising at least one of the plurality of peptide-tagged agents. In certain embodiments, the method may further comprise, for example, transporting the analyte complex to a detection zone of a lateral flow device. In certain embodiments, the analyte complex may comprise, for example, the analyte and at least one of the plurality of peptide-tagged agents. In certain embodiments, the detection zone may comprise, for example, a plurality of anti-peptide agents present, for example, at a ratio of at least 100:1, on a weight:weight basis, relative to the plurality of peptide-tagged agents. In certain embodiments, the plurality of anti-peptide agents may be capable of immobilizing at least one of the at least one of the plurality of peptide-tagged agents.

In certain embodiments, transporting may comprise, for example, bulk transport, passive transport, capillary action, wetting, wicking, or a combination of two or more thereof on a lateral flow device. In certain embodiments, the at least one of the at least one of the plurality of peptide-tagged agents may comprise, for example, a plurality of covalently conjugated peptide tags, wherein at least one of said covalently conjugated peptide tags may be configured, for example, to bind with at least one of the plurality of anti-peptide agents. In certain embodiments, the analyte complex may be formed on a portion of the lateral flow device. In certain embodiments, at least a portion of the analyte complex may not be formed on any portion of the lateral flow device. In certain embodiments, the method may further comprise, for example, releasing said plurality of peptide-tagged agents from a bound state on a portion of the lateral flow device prior to forming the analyte complex, for example by exposing said plurality of peptide-tagged agents to a sample and/or solvent. In certain embodiments, the method may further comprise, for example, introducing said plurality of peptide-tagged agents in a liquid to the lateral flow device. In certain embodiments, forming the analyte complex may further comprise, for example, exposing at least a portion of sample and at least a portion of the plurality of peptide-tagged agents to a solvent. In certain further embodiments, the at least a portion of the sample may be exposed to the solvent before, after, or coincident with the at least a portion of the plurality of peptide-tagged agents. In certain embodiments, the method may further comprise, for example, immobilizing the analyte complex to a portion of the lateral flow device. In certain embodiments, the method may further comprise, for example, immobilizing the analyte complex to a portion of the detection zone. In certain embodiments, the method may further comprise, for example, binding at least one of the at least one of the plurality of peptide-tagged agents to at least one of the plurality of anti-peptide agents. In certain further embodiments, the method may further comprise, for example, releasing said plurality of detectable agents from a bound state on a portion of the lateral flow device, for example by exposing said plurality of detectable agents to a sample and/or solvent. In certain other embodiments, the method may further comprise, for example, introducing said plurality of detectable agents in a liquid to the lateral flow device. In certain embodiments, the method may further comprise, for example, detecting said immobilized analyte complex. In certain further embodiments, the method may comprise, for example, detecting said immobilized analyte complex by detecting a detectable agent bound to the immobilized analyte complex.

In certain embodiments, the detecting may be accomplished in less than 2 hours, for example in less than 90 minutes, 80 minutes, 70 minutes, 60 minutes, 50 minutes, 40 minutes, 30 minutes, 20 minutes, 18 minutes, 16 minutes, 14 minutes, 12 minutes, 10 minutes, 9 minutes, 8 minutes, 7 minutes, 6 minutes, 5 minutes, 4 minutes, 3 minutes, 2 minutes, or in less than 1 minute. In certain embodiments, the detecting may be accomplished in the range of between 1 minute and 20 minutes, for example in the range of between 1 minute and 5 minutes, 5 minutes and 15 minutes, 5 minutes and 10 minutes, or in the range of between 3 minutes and 8 minutes. In certain embodiments, the detecting may require at least 10 minutes, for example at least 15 minutes, 20 minutes, 30 minutes, 45 minutes, or at least 1 hour.

Certain embodiments may provide, for example, a method, comprising: (i) exposing a sample comprising a pre-determined analyte to a plurality of peptide-tagged agents to form an analyte complex, said analyte complex comprising the pre-determined analyte and at least one of the plurality of peptide-tagged agents; and (ii) transporting the analyte complex to a detection zone of a lateral flow device, said detection zone comprising a plurality of anti-peptide agents present at a ratio of at least 100:1, on a weight:weight basis, relative to the plurality of peptide-tagged agents, said plurality of anti-peptide agents capable of immobilizing at least one of the at least one of the plurality of peptide-tagged agents.

Certain embodiments may provide a method. In certain embodiments, the method may be configured to detect at least two analytes, for example at least two analytes, at least three analytes, at least four analytes, at least five analytes, at least six analytes, at least seven analytes, at least eight analytes, at least nine analytes, or detecting at least ten analytes. In certain embodiments, the method may be configured to detect up to ten analytes. In certain embodiments, the method may comprise, for example, exposing a sample that may comprise a first analyte and a second analyte to a plurality of peptide-tagged agents to form at least a first analyte complex and a second analyte complex. In certain embodiments, the method may further comprise, for example, transporting at least the first analyte complex and the second analyte complex to a detection zone of a lateral flow device. In certain embodiments, the first analyte complex may comprise, for example, the first analyte and at least a first one of the plurality of peptide-tagged agents. In certain embodiments, the second analyte complex may comprise, for example, the second analyte and at least a second one of the plurality of peptide-tagged agents. In certain embodiments, the detection zone may comprise, for example, a plurality of anti-peptide agents present, for example, at a ratio of at least 100:1, on a weight:weight basis, relative to the plurality of peptide-tagged agents. In certain embodiments, the plurality of anti-peptide agents may be capable of immobilizing at least one of the at least one of the plurality of peptide-tagged agents. In certain embodiments, the plurality of anti-peptide agents may be configured to immobilize the first analyte complex and the second analyte complex.

In certain embodiments, the first one of the plurality of peptide-tagged agents may comprise a first type of peptide tag and a first type of capture agent. In certain embodiments, the second one of the plurality of peptide-tagged agents may comprise a second type of peptide tag and a second type of capture agent. In certain further embodiments, the first type of peptide tag and the second type of peptide tag may be different types of peptide tags and/or sequences of peptides. In certain further embodiments, the first type of capture agent and the second type of capture agent may be different types of capture agents, for example different types of antibodies. In certain embodiments, the plurality of anti-peptide agents may comprise at least two types of anti-peptide agents. In certain further embodiments, the plurality of anti-peptide agents may comprise a first type of anti-peptide agent which may be configured to bind with the first type of peptide tag, and the plurality of anti-peptide agents may further comprise a second type of anti-peptide agent which may be configured to bind with the second type of peptide tag. In certain further embodiments, the first type of anti-peptide agent may be a specific binding partner with the first type of peptide tag, and the second type of anti-peptide agent may be a specific binding partner with the second type of peptide tag.

Certain embodiments may provide, for example, a method of detecting at least two analytes, comprising: (i) exposing a sample comprising a first analyte and a second analyte to a plurality of peptide-tagged agents to form a first analyte complex and a second analyte complex, (a) said first analyte complex comprising the first analyte and at least a first one of the plurality of peptide-tagged agents; and (b) said second analyte complex comprising the second analyte and at least a second one of the plurality of peptide-tagged agents; and (ii) transporting the first analyte complex and the second analyte complex to a detection zone of a lateral flow device, said detection zone comprising a plurality of anti-peptide agents present at a ratio of at least 100:1, on a weight:weight basis, relative to the plurality of peptide-tagged agents, said plurality of anti-peptide agents configured to immobilize the first analyte complex and the second analyte complex.

Certain embodiments, for example, may provide a method. In certain embodiments, the method may comprise, for example, exposing a sample that may comprise a first analyte and a second analyte to a plurality of first peptide-tagged agents and to a plurality of second peptide-tagged agents to form a first analyte complex and a second analyte complex. In certain embodiments, the method may further comprise, for example, transporting the first analyte complex and the second analyte complex to a detection zone of a lateral flow device. In certain embodiments, the first analyte complex may comprise, for example, the first analyte and at least one of the plurality of first peptide-tagged agents. In certain embodiments, the second analyte complex may comprise, for example, the second analyte and at least one of the plurality of second peptide-tagged agents. In certain embodiments, for example, the detection zone may comprise, for example, a plurality of first anti-peptide agents present, for example, at a ratio of at least 100:1, on a weight:weight basis, relative to the plurality of first peptide-tagged agents. In certain further embodiments, for example, the detection zone may comprise, for example, a plurality of second anti-peptide agents present, for example, at a ratio of at least 100:1, on a weight:weight basis, relative to the plurality of second peptide-tagged agents. In certain embodiments, the plurality of first anti-peptide agents may be configured to immobilize the first analyte complex. In certain embodiments, the plurality of second anti-peptide agents may be configured to immobilize the second analyte complex.

In certain embodiments, at least a portion of the plurality of first anti-peptide agents may be bound in a first sub-zone of the detection zone. In certain embodiments, at least a portion of the plurality of second anti-peptide agents may be bound in a second sub-zone of the detection zone. In certain embodiments, the first sub-zone and the second sub-zone may be overlapping. In certain embodiments, the first sub-zone and the second sub-zone may be disjoint. In certain embodiments, a portion of the sample may migrate through the first sub-zone before at least a portion of said portion migrates through the second sub-zone.

Certain embodiments may provide, for example, a method of detecting at least two analytes, comprising: (i) exposing a sample comprising a first analyte and a second analyte to a plurality of first peptide-tagged agents and to a plurality of second peptide-tagged agents to form a first analyte complex and a second analyte complex, (a) said first analyte complex comprising the first analyte and at least one of the plurality of first peptide-tagged agents; and (b) said second analyte complex comprising the second analyte and at least one of the plurality of second peptide-tagged agents; and (ii) transporting the first analyte complex and the second analyte complex to a detection zone of a lateral flow device, said detection zone comprising: (a) a plurality of first anti-peptide agents present at a ratio of at least 100:1, on a weight:weight basis, relative to the plurality of first peptide-tagged agents, said plurality of first anti-peptide agents configured to immobilize the first analyte complex; and (b) a plurality of second anti-peptide agents configured to immobilize the second analyte complex.

Certain embodiments may provide a multi-purpose lateral flow test kit. In certain embodiments, the multi-purpose lateral flow kit may comprise, for example, a universal receiver, wherein the universal receiver may be configured, for example, to receive any one of a plurality of permeable members and, optionally, to bring said any one of the plurality of permeable members into fluid communication with a detection zone. In certain embodiments, the detection zone may comprise, for example, at least one anti-peptide agent. In certain embodiments, the multi-purpose lateral flow kit may further comprise a first permeable member of the plurality of permeable members. In certain embodiments, the multi-purpose lateral flow kit may further comprise a second permeable member of the plurality of permeable members. In certain embodiments, the first permeable member may comprise, for example, a first peptide-tagged agent that may be capable of binding with a first pre-determined type of analyte. In certain embodiments, the second permeable member may comprise, for example, a second peptide-tagged agent that may be capable of binding with a second pre-determined type of analyte. In certain embodiments, each of the first and second peptide-tagged agents may be a binding parter with at least one of the at least one anti-peptide agent.

Certain embodiments may provide, for example, a multi-purpose lateral flow test kit, comprising: (i) a universal receiver, said universal receiver configured to receive any one of a plurality of permeable members and to bring said any one of the plurality of permeable members into fluid communication with a detection zone, said detection zone comprising at least one anti-peptide agent; (ii) a first permeable member of the plurality of permeable members, comprising a first peptide-tagged agent capable of binding with a first pre-determined analyte; and (iii) a second permeable member of the plurality of permeable members, comprising a second peptide-tagged agent capable of binding with a second pre-determined analyte, wherein each of the first and second peptide-tagged agents is a binding partner with at least one of the at least one anti-peptide agent.

Certain embodiments may provide a divergent flow device for detecting an analyte. In certain embodiments, the divergent flow device may comprise a plurality of peptide-tagged agents. In certain embodiments, the divergent flow device may comprise a central release zone that may be configured to introduce said plurality of peptide-tagged agents into a divergent flow path. In certain embodiments, a plurality of anti-peptide agents may be present at a ratio of at least 100:1, on a weight:weight basis, relative to the plurality of peptide-tagged agents. In certain embodiments, the plurality of anti-peptide agents may be positioned in the divergent flow path about the central release zone. In certain embodiments, the divergent flow device may be configured to immobilize at least one of the plurality of peptide-tagged agents. In certain embodiments, the divergent flow path may be a radial flow path.

Certain embodiments may provide, for example, a divergent flow device, comprising: (i) a plurality of peptide-tagged agents; (ii) a central release zone configured to introduce said plurality of peptide-tagged agents into a divergent flow path; and (iii) a plurality of anti-peptide agents present at a ratio of at least 100:1, on a weight:weight basis, relative to the plurality of peptide-tagged agents, said plurality of anti-peptide agents: (a) positioned in the divergent flow path about the central release zone; and (b) capable of immobilizing at least one of the plurality of peptide-tagged agents.

Certain embodiments may provide a divergent flow device. In certain embodiments, the divergent flow device may be configured to detect a plurality of different types of analytes, for example at least two different types of analytes, at least three different types of analytes, at least four different types of analytes, at least five different types of analytes, at least six different types of analytes, at least seven different types of analytes, at least eight different types of analytes, at least nine different types of analytes, or configured to detect at least ten different types of analytes. In certain embodiments, the divergent flow device may be configured to detect up to ten different types of analytes. In certain embodiments, the divergent flow device may comprise a plurality of peptide-tagged agents.

In certain embodiments, the divergent flow device may comprise a central sample zone that may be configured to communicate a sample containing a plurality of analytes into a divergent flow path. In certain further embodiments, the divergent flow path may comprise a first sub-path. In certain embodiments, the divergent flow path may further comprise a second sub-path. In certain embodiments, the first sub-path may comprise a plurality of first peptide-tagged agents, at least a portion of said plurality of first peptide-tagged agents capable of selectively binding with a first type of analyte. In certain further embodiments, the first subpath may further comprise a plurality of first anti-peptide agents, at least a portion of said plurality of first anti-peptide agents capable of selectively binding with at least a portion of said plurality of first peptide-tagged agents. In certain further embodiments, the plurality of first anti-peptide agents may be present at a ratio of at least 100:1, on a weight:weight basis, relative to the plurality of first peptide-tagged agents. In certain further embodiments, the plurality of first anti-peptide agents may be configured to immobilize at least one of the plurality of first peptide-tagged agents. In certain embodiments, the second sub-path may comprise a plurality of second peptide-tagged agents, at least a portion of said plurality of second peptide-tagged agents capable of selectively binding with a second type of analyte. In certain further embodiments, the second subpath may further comprise a plurality of second anti-peptide agents, at least a portion of said plurality of second anti-peptide agents capable of selectively binding with at least a portion of said plurality of second peptide-tagged agents. In certain further embodiments, the plurality of second anti-peptide agents may be present at a ratio of at least 100:1, on a weight:weight basis, relative to the plurality of second peptide-tagged agents. In certain further embodiments, the plurality of second anti-peptide agents may be configured to immobilize at least one of the plurality of second peptide-tagged agents.

In certain embodiments, the divergent flow path may flow in a radial direction defined by the divergent flow device. In certain further embodiments, the first sub-path may be defined by a section of the divergent flow path. In certain further embodiments, the second sub-path may be defined by a further section of the divergent flow path. In certain embodiments, the divergent flow path may be defined by a permeable sub-assembly of the of the divergent flow device.

In certain embodiments, the first sub-path and the second sub-path are in fluid communication. In certain embodiments, the first sub-path and the second sub-path are not in fluid communication.

Certain embodiments may provide, for example, a divergent flow device for detecting a plurality of analytes, comprising: a central sample zone configured to communicate a sample containing a plurality of analytes into a divergent flow path, said divergent flow path comprising: (i) a first sub-path comprising: (a) a plurality of first peptide-tagged agents; and (b) a first plurality of anti-peptide agents present at a ratio of at least 100:1, on a weight:weight basis, relative to the plurality of first peptide-tagged agents, said plurality of first anti-peptide agents capable of immobilizing at least one of the plurality of first peptide-tagged agents; and (ii) a second sub-path comprising: (a) a plurality of second peptide-tagged agents; and (b) said second sub-path comprising a second plurality of anti-peptide agents capable of immobilizing at least one of the plurality of second peptide-tagged agents.

Certain embodiments may provide a divergent flow device for detecting a plurality of analytes. In certain embodiments, the divergent flow device may comprise a central sample zone that may be configured to release a sample containing the plurality of analytes into a first divergent flow path and a second divergent flow path. In certain embodiments, the divergent flow device may further comprise a first release zone in the first divergent flow path that may be configured to release a plurality of first peptide-tagged agents into the first divergent flow path. In certain embodiments, the divergent flow device may further comprise a second release zone in the second divergent flow path that may be configured to release a plurality of second peptide-tagged agents into the second divergent flow path. In certain embodiments, the divergent flow device may further comprise a plurality of first anti-peptide agents that may be present at a ratio of at least 100:1, on a weight:weight basis, relative to the plurality of first peptide-tagged agents. In certain further embodiments, the plurality of first anti-peptide agents may be positioned in the first divergent flow path downstream of the first release zone. In certain further embodiments, the plurality of first anti-peptide agents may be configured to immobilize at least one of the plurality of first peptide-tagged agents. In certain embodiments, the divergent flow device may further comprise a plurality of second anti-peptide agents. In certain further embodiments, the plurality of second anti-peptide agents may be positioned in the second divergent flow path downstream of the second release zone. In certain further embodiments, the plurality of second anti-peptide agents may be configured to immobilize at least one of the plurality of second peptide-tagged agents.

In certain embodiments, the plurality of first anti-peptide agents may be positioned in a first detection zone. In certain embodiments, the plurality of second anti-peptide agents may be positioned in a second detection zone. In certain further embodiments, the first detection zone and the second detection zone may be in fluid communication. In certain further embodiments, the first detection zone and the second detection zone may not be in fluid communication. In certain further embodiments, the first detection zone and the second detection zone may be in different sections of the divergent flow device. In certain embodiments, the first divergent flow path and the second divergent flow path may be in different sections of the divergent flow device.

Certain embodiments may provide, for example, a divergent flow device, comprising: (i) a central sample zone configured to release a sample containing a plurality of analytes into a first divergent flow path and a second divergent flow path; (ii) a first release zone in the first divergent flow path configured to release a plurality of first peptide-tagged agents into the first divergent flow path; (iii) a second release zone in the second divergent flow path configured to release a plurality of second peptide-tagged agents into the second divergent flow path; (iv) a plurality of first anti-peptide agents present at a ratio of at least 100:1, on a weight:weight basis, relative to the plurality of first peptide-tagged agents, said plurality of first anti-peptide agents: (a) positioned in the first divergent flow path downstream of the first release zone; and (b) capable of immobilizing at least one of the plurality of first peptide-tagged agents; and (v) a plurality of second anti-peptide agents positioned in the second divergent flow path downstream of the second release zone and the second release zone, said plurality of second anti-peptide agents capable of immobilizing at least one of the plurality of second peptide-tagged agents.

Certain embodiments may provide a method of making a lateral flow device. In certain embodiments, the method of making may comprise striping a plurality of peptide-tagged agents on a first portion of a permeable assembly. In certain embodiments, the method of making may comprise binding a plurality of anti-peptide agents to a second portion of the permeable assembly. In certain embodiments, the plurality of anti-peptide agents may be present on the test strip at a ratio of at least 100:1, on a weight:weight basis, relative to the plurality of peptide-tagged agents. In certain embodiments, the second portion of the permeable assembly may be in fluid communication with the first portion of the permeable assembly.

Certain embodiments may provide, for example, a method of making a lateral flow device, comprising: (i) binding a plurality of anti-peptide agents to a portion of a permeable assembly; (ii) striping a plurality of peptide-tagged agents on a second portion of the permeable assembly, said second portion of the permeable assembly in fluid communication with the first portion of the permeable assembly, at least one of said plurality of peptide-tagged agents forming a specific binding pair with at least one of said plurality of anti-peptide agents, wherein said plurality of anti-peptide agents are present on the test strip at a ratio of at least 100:1, on a weight:weight basis, relative to the peptide-tagged agent.

Certain embodiments may provide a method of making a plurality of lateral flow devices. In certain embodiments, the method may comprise preparing a quantity of universal detection members. In certain embodiments, the method may further comprise preparing a quantity of release members. In certain embodiments, the method may further comprise, for example, assembling into fluid communication each of the quantity of release members with a different one of the quantity of universal detection members. In certain embodiments, each of the universal detection members may have a common type of anti-peptide immobilization agent. In certain embodiments, preparing the quantity of universal detection members may comprise, for example, immobilizing a different plurality of the common type of anti-peptide agent onto one or more sheets, for example one sheet, and partitioning the one or more sheets to form the plurality of universal detection members. In certain embodiments, preparing the quantity of release members may comprise, for example, striping a plurality of a different type of peptide-tagged agent onto one or more sheets, for example one seet, and partitioning the one or more sheets to form the plurality of release members. In certain further embodiments, each of the different type of peptide-tagged agent may form a specific binding pair with the common type of anti-peptide agent. In certain embodiments, each of the different plurality of the common type of anti-peptide agent may be present at a ratio of at least 100:1, on a weight:weight basis, relative to each of the plurality of the different type of peptide-tagged agent. In certain embodiments, at least one of the different plurality of the common type of anti-peptide agent may be present at a ratio of at least 100:1, on a weight:weight basis, relative to at least one of the plurality of the different type of peptide-tagged agent.

In certain embodiments, each one of the plurality of a different type of peptide-tagged agent may comprise a different plurality of peptide tags. In certain further embodiments, at least one of each of the different plurality of peptide tags may be a type of peptide tag which may be a binding partner with the common type of anti-peptide agent.

In certain embodiments, an apparatus, lateral flow device, assembly, permeable assembly, divergent flow device, lateral flow test kit, or multi-purpose lateral flow test kit may comprise a control zone. In certain further embodiments, the control zone may be configured to provide an indication that the apparatus has functioned. In certain further embodiments, the control zone may be configured to non-selectively capture species present in the sample, for example proteins, antibodies, macromolecules, particles, and the like. In certain embodiments, the control zone may comprise, for example, protein A and/or protein G.

Certain embodiments may provide, for example, a method of making a plurality of lateral flow devices, comprising: (i) preparing a quantity of universal detection members having a common type of anti-peptide immobilization agent, comprising: immobilizing a different plurality of the common type of anti-peptide agent to each of the plurality of universal detection members; (ii) preparing a quantity of release members, comprising: striping a plurality of a different type of peptide-tagged agent on each of the plurality of release members, each of the different type of peptide tagged agent forming a specific binding pair with the common type of anti-peptide agent; and (iii) assembling into fluid communication each of the quantity of release members with a different one of the quantity of universal detection members, wherein at least one of the different plurality of the common type of anti-peptide agent is present at a ratio of at least 100:1, on a weight:weight basis, relative to at least one of the plurality of the different type of peptide-tagged agent.

Certain embodiments may provide, for example, a lateral flow device, comprising: (i) a flow path defined by a permeable sub-assembly of the lateral flow device; (ii) a release zone comprising: (a) a plurality of first peptide-tagged agents, said release zone configured to release at least a portion of said plurality of first peptide-tagged agents into the flow path; and (b) a plurality of second peptide-tagged agents, said release zone configured to release at least a portion of said plurality of second peptide-tagged agents into the flow path; and (iii) a detection zone comprising: (a) a plurality of first anti-peptide agents present in the flow path at a ratio of at least 100:1, on a weight:weight basis, relative to the plurality of first peptide-tagged agents, wherein at least one of said plurality of first anti-peptide agents is a binding partner with at least one of the configured to be released at least a portion of said plurality of first peptide-tagged agents; and (b) a plurality of second anti-peptide agents present in the flow path at a ratio of at least 100:1, on a weight:weight basis, relative to the plurality of second peptide-tagged agents, wherein at least one of said plurality of second anti-peptide agents is a binding partner with at least one of the configured to be released at least a portion of said plurality of second peptide-tagged agents.

Certain embodiments may provide, for example, an assay platform for detecting a plurality of analytes, comprising: (i) a permeable release zone configured to transport a sample comprising a first analyte and a second analyte, said permeable zone comprising: (a) a plurality of first peptide-tagged agents, at least a portion of said plurality of first peptide-tagged agents capable of forming a first analyte complex comprising the first analyte; and (b) a plurality of second peptide-tagged agents, at least a portion of said plurality of second peptide-tagged agents capable of forming a second analyte complex comprising the second analyte; and (ii) a permeable detection zone configured to receive at least a portion of transported sample, the permeable detection zone comprising: (a) a plurality of first anti-peptide agents present at a ratio of at least 100:1, on a weight:weight basis, relative to the plurality of first peptide-tagged agents, said plurality of first anti-peptide agents configured to immobilize at least one of the at least a portion of said plurality of first peptide-tagged agents; and (b) a plurality of second anti-peptide agents configured to immobilize at least one of the at least a portion of said plurality of second peptide-tagged agents.

### DETAILED DESCRIPTION OF THE DRAWINGS

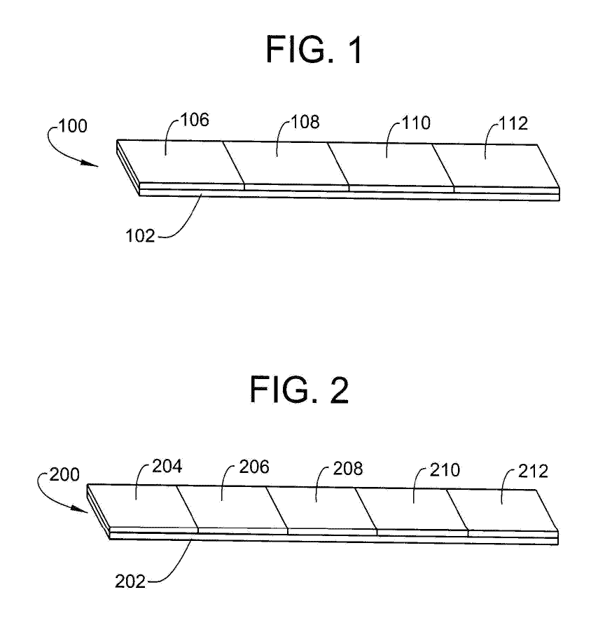

FIG. 1: Schematic view of lateral flow detection apparatus.

FIG. 2: Schematic view of solvent-driven lateral flow detection apparatus.

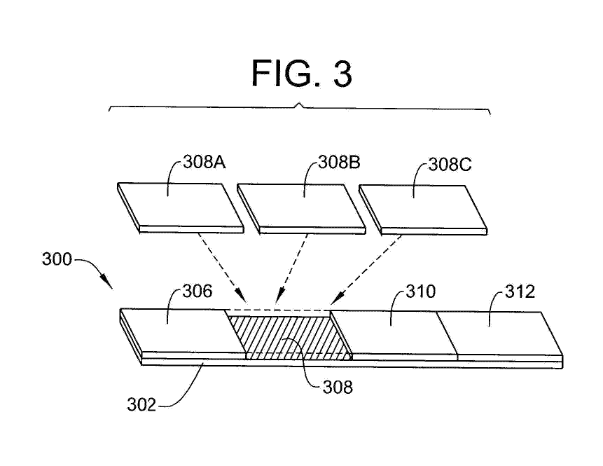

FIG. 3: Schematic view of multi-purpose lateral flow test kit.

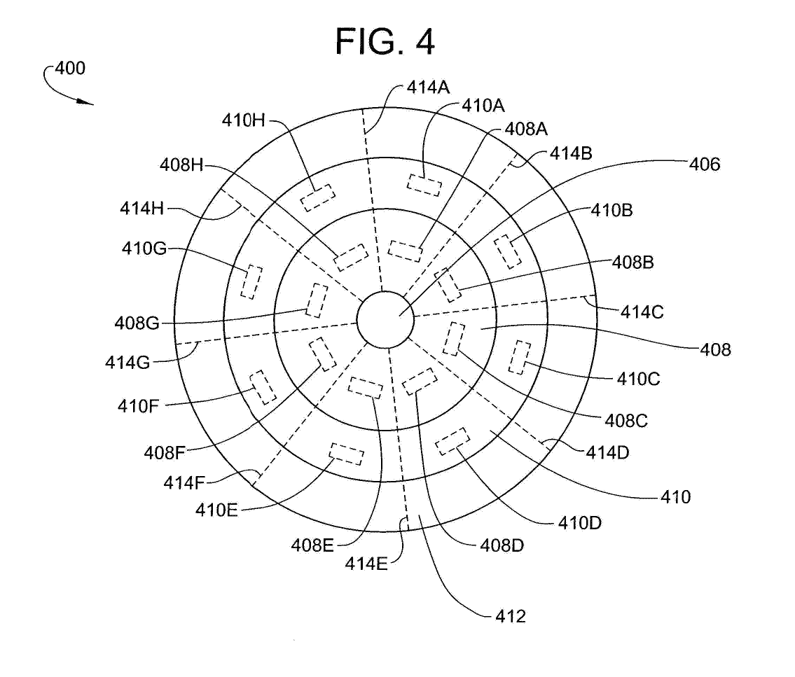

FIG. 4: Schematic view of radial flow detection apparatus.

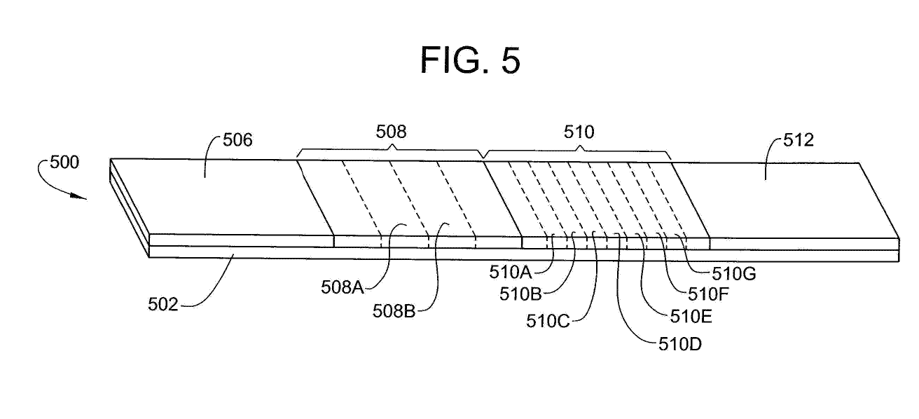

FIG. 5: Schematic view of multi-analyte lateral flow detection apparatus.

### DETAILED DESCRIPTION OF THE INVENTION

Certain embodiments provide lateral flow apparatuses and systems to detect one or more analytes, for example: assay platforms, lateral flow devices, assemblies, single-purpose kits, multi-purpose kits, and divergent flow devices. Other types of lateral flow apparatuses are contemplated herein. Certain embodiments provide lateral flow methods to detect one or more analytes, including, for example, methods of detection, methods of making lateral flow apparatuses, and methods of making lateral flow kits. Other types of lateral flow methods are contemplated herein.

Suitable analytes may include any analyte to which a peptide-tagged agent is able to bind (for example the peptide-tagged agent may be designed or selected to bind a pre-determined analyte). In certain embodiments, the analyte may be a predetermined analyte, for example an analyte for which a specific type of peptide-tagged agent is selected. In certain embodiments, the analyte may comprise, for example, a protein, microbe or a fragment thereof, a virus or a fragment thereof, a peptide, a biomarker, an antibody (for example, an antibody to an infectious agent) or a fragment thereof, a nucleic acid, a macromolecule, a small molecule, a drug, a hormone (for example, human chorionic gonadotrophin) or a fragment thereof, a hapten or a fragment thereof, etc. In certain embodiments, the analyte may be any analyte which has heretofore been assayed using known immunoassay procedures, or known to be detectable by such procedures (see, for example, “The Immunoassay Handbook,” Fourth Ed., D. Wild ed., Elsevier Ltd. (2013)). Other types of analytes are also contemplated herein.

Protein analytes may include, for example, a phosphoprotein. A range of phosphoproteins are known including, for example, phosphorylated ERK, S6 p240/44, AKT pT308 or AKT pS473. In certain embodiments, the analyte may comprise, for example, a component of a cell signaling pathway, a cytokine, or a tumour suppressor. In certain embodiments, the analyte may comprise, for example, a protein that is phosphorylated, methylated, or glycosylated, etc. In certain embodiments, the analyte may comprise, for example, phospho-ERK 1/2; total ERK 1/2; phospho-Akt 1/2/3; total Akt 1/2/3; phospho-NF-Kβ p65; total NF-Kβ p65; phospho-l-kB; total-kBP; phospho-STAT3; total STAT3; phospho-STAT5 A/B; phospho-JNK 1/2/3; total JNK 1/2/3; phospho-p38 MAPK; total p38 MAPK; phospho-p53; total p53; phospho-p70S6K; total p70S6K; and GAPDH. In other embodiments, the protein may comprise, for example, an acute phase protein, for example a C-reactive protein, haptoglobin, hemopexin, alpha-1 acid glycoprotein, clusterin, alpha-2-macroglobulin, serum amyloid A (SAA) or serum amyloid P (SAP) in species including but not limited to human, mouse, rat, rabbit, cat, dog, pig, cow, chicken, and monkey. In certain embodiments, the protein may comprise, for example, a particular form or state of a protein, an endogenous protein, or a transfected protein.

In certain embodiments, the analyte may comprise, for example, a biomarker, for example a cancer biomarker (for example a prostate cancer biomarker or a bowel cancer biomarker) or a diabetic kidney disease biomarker. In certain embodiments, the analyte may comprise, for example, an analyte useful for detection in plant or contaminant diagnostics. In certain other embodiments, the analyte may comprise, for example, an analyte useful for detection in human or veterinary diagnostics. In certain embodiments, the analyte may be capable of migrating, dissolving, disconnecting, or diffusing from the sample into a solvent. Suitable solvents may comprise, for example, water, an alcohol, a buffered solution, a pH-balanced solution, an organic solvent, or a combination of two or more thereof.

In certain embodiments, the concentration of the analyte or any one of a plurality of analytes may be in a range of 0-100 ng/mL, for example, between 1-100 ng/mL, between 1-50 ng/mL, between 1-25 ng/mL, between 10-50 ng/mL, of in a range of 1-10 ng/mL. In certain embodiments, the concentration of the analyte, the one or more analytes may be below 1 ng/mL. In certain embodiments, the concentration of the analyte or any one of a plurality of analytes may be above 50 ng/mL. In certain embodiments, the concentration of the analyte or any one of a plurality of analytes may be 100 ng/mL or less, for example 50 ng/mL or less, 10 ng/mL or less, 1 ng/mL or less, 100 pg/mL or less, or 10 pg/mL or less, 1 pg/mL or less, 100 fg/mL or less, 10 fg/mL or less, or 1 fg/mL or less. In certain embodiments, the concentration of the analyte or any one of a plurality of analytes may be greater than 10 ng/mL, greater than 1 ng/mL, greater than 100 pg/mL or, greater than 10 pg/mL, greater than 1 pg/mL, greater than 100 fg/mL, greater than 10 fg/mL, or greater than 1 fg/mL. In certain embodiments, the concentration of the analyte or any one of a plurality of analytes may be in the range of 1 fg/mL to 100 ng/mL, for example 1 fg/mL to 10 ng/mL, 1 fg/mL to 1 ng/mL, 10 fg/mL to 100 ng/mL, 10 fg/mL to 10 ng/mL, 10 fg/mL to 1 ng/mL, 100 fg/mL to 100 ng/mL, 100 fg/mL to 10 ng/mL, 100 fg/mL to 1 ng/mL, 1 pg/mL to 100 ng/mL, 1 pg/mL to 10 ng/mL, or in the range of 1 pg/mL to 1 ng/mL.

In certain embodiments, the concentration of the analyte or any one of a plurality of analytes may be in a range of 0 mIU/mL (milli-International Units per milli-liter) to 100 mIU/mL, for example 0.25 mIU/mL to 0.5 mIU/mL, 0.5 mIU/mL to 1 mIU/mL, 1 mIU/mL to 2 mIU/mL, 2 mIU/mL to 5 mIU/mL, 5 mIU/mL to 10 mIU/mL, 10 mIU/mL to 15 mIU/mL, 15 mIU/mL to 25 mIU/mL, 25 mIU/mL to 50 mIU/mL, or in a range of 50 mIU/mL to 100 mIU/mL. In certain embodiments, the concentration of the analyte or any one of a plurality of analytes may be less than 100 mIU/mL, less than 50 mIU/mL, less than 25 mIU/mL, less than 15 mIU/mL, less than 10 mIU/mL, less than 5 mIU/mL, less than 2 mIU/mL, less than 1 mIU/mL, less than 0.5 mIU/mL, or less than 0.25 mIU/mL.

In certain embodiments, the analyte may have a molecular weight in a range of 10 Da to 10,000,000 kDa, for example a molecular weight in a range of 10 Da to 25 Da, 25 Da to 75 Da, 75 Da to 100 Da, 100 Da to 200 Da, 200 Da to 300 Da, 300 Da to 500 Da, 500 Da to 750 Da, 750 Da to 200 Da, 100 Da to 1 kDa, 1 kDa to 10 kDa, 10 kDa to 15 kDa, 15 kDa to 20 kDa, 20 kDa to 25 kDa, 25 kDa to 50 kDa, 50 kDa to 75 kDa, 75 kDa to 100 kDa, 100 kDa to 125 kDa, 125 kDa to 150 kDa, 150 kDa to 175 kDa, 175 kDa to 200 kDa, 200 kDa to 250 kDa, 250 kDa to 500 kDa, 500 kDa to 1,000 kDa, 1,000 kDa to 5,000 kDa, 5,000 kDa to 10,000 kDa, 10,000 KDa to 1,000,000 kDa, 1,000,000 kDa to 10,000,000 kDa, 75 kDa to 400 kDa, 100 kDa to 200 kDa, or a molecular weight in a range of 125 kDa to 175 kDa.

Certain embodiments may provide an assay platform for detecting at least one type of an analyte in a sample, optionally in the presence of one or more type of non-analyte antibody. In certain embodiments, for example, the assay platform may comprise, for example, at least one type of peptide-tagged agent and at least one type of anti-peptide tag. In certain embodiments, the affinity, or rate constant, for binding one of the at least one type of anti-peptide agent to one of the at least one type of peptide-tagged agent may be approximately the same as the affinity, or rate constant, for binding the one of the at least one type of anti-peptide agent to any of the one or more type of non-analyte antibody. In certain embodiments, the affinity, or rate constant, for binding one of the at least one type of anti-peptide agent to one of the at least one type of peptide-tagged agent may be at least 2 times larger than the affinity, or rate constant, for binding the one of the at least one type of anti-peptide agent to any of the one or more type of non-analyte antibody, for example at least 5, 10, 25, 50, 75, or at least 100 times larger. In certain embodiments, the affinity, or rate constant, for binding one of the at least one type of anti-peptide agent to one of the at least one type of peptide-tagged agent may be in the range of 2-1000 times larger than the affinity, or rate constant, for binding the one of the at least one type of anti-peptide agent to any of the one or more type of non-analyte antibody, for example in the range of 2-500, 2-100, 2-10, 10-250, 20-100, or in the range of 5-50 times larger.

In certain embodiments, the dissociation constant between one of the at least one type of anti-peptide agent and one of the at least one type of peptide-tagged agent may be approximately the same as the dissociation constant between one of the at least one type of anti-peptide agent and any of the one or more type of non-analyte antibody. In certain embodiments, the dissociation constant between one of the at least one type of anti-peptide agent and one of the at least one type of peptide-tagged agent may be at least 2 times larger than the dissociation constant between one of the at least one type of anti-peptide agent and any of the one or more type of non-analyte antibody, for example at least 5, 10, 25, 50, 75, or at least 100 times larger. In certain embodiments, the dissociation constant between one of the at least one type of anti-peptide agent and one of the at least one type of peptide-tagged agent may be in the range of 2-1000 times larger than the dissociation constant between one of the at least one type of anti-peptide agent and any of the one or more type of non-analyte antibody, for example in the range of 2-500, 2-100, 2-10, 10-250, 20-100, or in the range of 5-50 times larger.

In certain embodiments, one of the at least one type of peptide-tagged agent may form a first specific binding pair with one of one or more pre-determined type of analyte, wherein said specific binding pair may have a dissociation constant (Kd) of greater than 10⁻⁶M. In further embodiments, said first specific binding pair may have a Kd of greater than 10⁻⁷M, 10⁻⁸M or 10⁻⁹M. In certain embodiments, said first specific binding pair may have a dissociation constant (Kd) in the range from 10⁻⁸M to 10⁻¹²M. In certain embodiments, the one of the at least one type of peptide-tagged agent may form a second specific binding pair with one of the at least one type of anti-peptide agent, wherein said second specific binding pair may have a dissociation constant (Kd) of greater than 10⁻⁶M. In further embodiments, said second second specific binding pair may have a Kd of greater than 10⁻⁷M, 10⁻⁸M or 10⁻⁹M. In certain embodiments, said second specific binding pair may have a dissociation constant (Kd) in the range from 10⁻⁸M to 10⁻¹²M. In certain embodiments, the first specific binding pair may have a Kd in the range of 10⁻⁸M to 10⁻¹²M and the second specific binding pair may have a Kd in the range of greater than 10⁻⁶M. In certain embodiments, the second specific binding pair may have a Kd in the range of 10⁻⁸M to 10⁻¹²M and the first specific binding pair may have a Kd in the range of greater than 10⁻⁶M.

In certain embodiments, one of the at least one type of peptide-tagged agent may be present in the assay platform in an amount in the range of between 1 ng and 50 ng, for example in the range of between 3 ng and 30 ng, 4 ng and 20 ng, 4 ng and 15 ng, 5 ng and 10 ng, or in the range of between 5 ng and 8 ng, for example 6.5 ng. In certain embodiments, one of the least one type of peptide-tagged agent may be present in the assay platform in an amount of less than 10 ng, for example less than 9 ng, 8 ng, 7.5 ng, 7 ng, 6.5 ng, 6 ng, 5.5 ng, 5 ng, 4.5 ng, 4 ng, 3.5 ng, 3 ng, 2.5 ng, or in an amount of less than 2 ng.

In certain embodiments, one of the at least one type of peptide-tagged agent may be present in the assay platform in an amount in the range of between 10 ng/cm² and 500 ng/cm² of test strip area striped by the one of the at least one type of peptide-tagged agent, for example in the range of between 30 ng/cm² and 300 ng/cm², 40 ng/cm² and 200 ng/cm², 40 ng/cm² and 150 ng/cm², 50 ng/cm² and 100 ng/cm², or in the range of between 50 ng/cm² and 80 ng/cm², for example 65 ng/cm² of test strip area striped by the one of the at least one type of peptide-tagged agent. In certain embodiments, one of the at least one type of peptide-tagged agent may be present in the assay platform in an amount of less than 100 ng/cm² of test strip area striped by the one of the at least one type of peptide-tagged agent, for example less than 90 ng/cm², 80 ng/cm², 75 ng/cm², 70 ng/cm², 65 ng/cm², 60 ng/cm², 55 ng/cm², 50 ng/cm², 45 ng/cm², 40 ng/cm², 35 ng/cm², 30 ng/cm², 25 ng/cm², or in an amount of less than 20 ng/cm² of test strip area striped by the one of the at least one type of peptide-tagged agent. In certain further embodiments, the test strip area striped by the one of the at least one type of peptide-tagged agent may have a surface area in the range of 0.01-5 cm², for example 0.1-2 cm², 0.15-2 cm², or a surface area in the range of 0.25-1 cm².

Certain embodiments may provide a lateral flow apparatus or system to detect an analyte. The lateral flow apparatus or system may comprise, for example, a plurality of peptide-tagged agents. In certain embodiments, the plurality of peptide-tagged agents may be capable of forming an analyte complex that may comprise at least one of the plurality of peptide-tagged agents and the analyte. In certain embodiments, the lateral flow apparatus or system may further comprise, for example, a plurality of anti-peptide agents present, for example, at a ratio of at least 100:1, on a weight:weight basis, relative to the plurality of peptide-tagged agents. In certain embodiments, the lateral flow apparatus or system may further comprise, for example, a plurality of detectable agents, and, optionally, the analyte complex may further comprise, for example, at least one of the plurality of detectable agents.

In certain embodiments, the sample may be any mixture, composition, or solution that may or may not contain the analyte. In certain assays, it may be useful, for example, to determine that an analyte may not be present in a sample. In certain embodiments, the sample may comprise, for example, a laboratory sample, a medical sample, a biological sample, a water sample, a food sample, an agricultural sample, a serum sample, a serum containing sample, a cell lysate, or a combination of two or more thereof. The sample may comprise, for example, urine, blood, or another bodily fluid of a human or animal.

In certain embodiments, the sample may be a liquid, for example an aqueous solution, a suspension, or a liquid mixture; a solid; a gel; or congealed matter. In certain embodiments, the sample may be dissolved, mixed, passively mixed, or diffused in a solvent. In certain embodiments, the sample may be pretreated, for example the sample may be precleared, concentrated, diluted, or processed to remove one or more components or impurities from the sample.

A peptide-tagged agent may comprise, for example, an agent that may be capable of binding with the analyte, for example an antibody, inclusive, for example, of a monoclonal antibody, a polyclonal antibody, a multivalent antibody, a chimeric antibody, a multispecific antibody, or an antibody fragment; an aptamer; an affimer; a protein; a protein receptor; a protein ligand; or a fusion protein, comprising, for example, an immunoglobulin fusion partner, a fusion partner that stabilizes a receptor or a ligand, or a fusion partner that provides a target for binding.

In certain embodiments, the peptide-tagged agent may have a molecular weight in a range of 100 Da to 10,000 kDa, for example a molecular weight in a range of 100 Da to 1 kDa, 1 kDa to 10 kDa, 10 kDa to 25 kDa, 25 kDa to 50 kDa, 50 kDa to 75 kDa, 75 kDa to 100 kDa, 100 kDa to 125 kDa, 125 kDa to 150 kDa, 150 kDa to 175 kDa, 175 kDa to 200 kDa, 200 kDa to 250 kDa, 250 kDa to 500 kDa, 500 kDa to 1,000 kDa, 1,000 kDa to 5,000 kDa, 5,000 kDa to 10,000 kDa, 75 kDa to 400 kDa, 100 kDa to 200 kDa, or a molecular weight in a range of 125 kDa to 175 kDa.

In certain embodiments, the peptide-tagged agent may be capable of binding to an epitope of the analyte, for example the peptide-tagged agent may comprise a complementary determinant region, or a portion thereof, that may be capable of binding with one or more epitopes of the analyte, for example a phospho-epitope. In certain embodiments, the analyte may be a target for the peptide-tagged agent. In certain embodiments, the peptide-tagged agent may be non-covalently bound to the analyte, for example releasably bound, partially bonded, hydrogen-bonded, ionically bonded, or a combination of two or more thereof.

In certain embodiments, the peptide-tagged agent may further comprise, for example, a plurality of peptide tags. In certain embodiments, the peptide-tagged agent may further comprise, for example, a plurality of a single type of peptide tag. In certain embodiments, the peptide-tagged agent may further comprise, for example, a plurality of two, three, or four types of peptide tags. In certain embodiments, the plurality of peptide tags may be attached, conjugated, and/or covalently bound to the peptide-tagged agent. In certain embodiments, the plurality of peptide tags may be bound to the peptide-tagged agent via cross-linking to the peptide-tagged agent. In certain further embodiments, the cross-linking may comprise, for example, covalent conjugation by primary amines. In certain embodiments, Recombinant DNA technology may be used to form a peptide-tagged agent comprising the plurality of peptide tags.

In certain embodiments, the plurality of peptide tags may comprise, for example, in a range of 1-50 or 2-50 peptide tags, such as 5-8, 3-7, 2-5, 10-15, 15-20, or in the range of 25-50 peptide tags. In certain embodiments, the plurality of peptide tags may comprise, for example, no more than 50 peptide tags, such as no more than 12, no more than 10, no more than 8, no more than 7, no more than 6, no more than 5, or no more than 3 peptide tags. In certain embodiments, the plurality of peptide tags may be 1, 2, 3, 4, 5, 6, 7, 8, 9, 10, 11, or 12 peptide tags.

Suitable peptide tags may include a FLAG-tag, for example DYKDDDDK (SEQ ID NO: 1); a peptide tag having the amino acid sequence KRITVEEALAHPYLEQYYDPTDE (SEQ ID NO: 2); a peptide tag having the amino acid sequence HHHHHH (SEQ ID NO: 3); a peptide tag having the amino acid sequence EQKLISEEDL (SEQ ID NO: 4); a peptide tag having the amino acid sequence YPYDVPDYA (SEQ ID NO: 5); a peptide tag having the amino acid sequence YTDIEMNRLGK (SEQ ID NO: 6); a peptide tag having the amino acid sequence QPELAPEDPED (SEQ ID NO: 7); a peptide tag having the amino acid sequence CDYKDDDDK (SEQ ID NO: 8) a FLAG octapeptide; a polypeptide protein tag; or a polypeptide sequence that does not include a plurality of consecutive amino acids with the same charge. In certain further embodiments, the suitable peptide tags may also be used in conjunction with other affinity tags, for example a polyhistidine tag (His-tag), HA-tag or myc-tag. In certain embodiments, at least a portion of the peptide tags may not be naturally occurring. In certain embodiments, the suitable peptide tags do not denature or inactivate the peptide-tagged agent to which they are attached. In certain embodiments, at least one of the suitable peptide tags is more hydrophilic than the FLAG-tag. In certain embodiments, at least a portion of the suitable peptide tags may be removed from the peptide-tagged agent to which they are attached by treatment with the specific proteinase, enterokinase (enteropeptidase). In certain embodiments, at least a portion of the suitable peptide tags may not be naturally occurring in an organism from which a sample is taken. In certain embodiments, suitable peptide tags may include amino acid sequences having no more than 100 amino acids, for example, no more than 50, no more than 30, no more than 20, no more than 15, no more than 12, no more than 10, no more than 9, or no more than 8 amino acids. In certain embodiments, suitable peptide tags may include amino acid sequences having in the range of 4-100 amino acids, for example in the range of 4-50, 4-30, 4-20, 6-15, 6-12, 8-20, 8-15, or in the range of 8-12 amino acids.

In certain embodiments, an anti-peptide agent may comprise, for example, an agent capable of binding with at least one of the plurality of peptide tags, for example at least 10%, 25%, 50%, 90%, or 95% of the plurality of peptide tags, such as for example, each of the plurality of peptide tags. In certain embodiments, a suitable anti-peptide agent may comprise, for example, an antibody, inclusive, for example, of a monoclonal antibody, a polyclonal antibody, a multivalent antibody, a chimeric antibody, a multispecific antibody, or an antibody fragment; an aptamer; an affimer; a protein; a protein receptor; a protein ligand; or a fusion protein, comprising, for example, an immunoglobulin fusion partner, a fusion partner that stabilizes a receptor or a ligand, or a fusion partner that provides a target for binding.

In certain embodiments, the anti-peptide agent may have a molecular weight in a range of 100 Da to 10,000 kDa, for example a molecular weight in a range of 100 Da to 1 kDa, 1 kDa to 10 kDa, 10 kDa to 25 kDa, 25 kDa to 50 kDa, 50 kDa to 75 kDa, 75 kDa to 100 kDa, 100 kDa to 125 kDa, 125 kDa to 150 kDa, 150 kDa to 175 kDa, 175 kDa to 200 kDa, 200 kDa to 250 kDa, 250 kDa to 500 kDa, 500 kDa to 1,000 kDa, 1,000 kDa to 5,000 kDa, 5,000 kDa to 10,000 kDa, 75 kDa to 400 kDa, 100 kDa to 200 kDa, or a molecular weight in a range of 125 kDa to 175 kDa.

In certain embodiments, at least one of the plurality of peptide tags may be a target for the anti-peptide agent. In certain embodiments, at least one of the plurality of peptide tags and the anti-peptide agent may be specific binding partners. In certain embodiments, the anti-peptide agent may comprise, for example, a binding region highly selective for at least one of the peptide tags, or a portion of at least one of the peptide tags, such as, for example for a peptide tag having the amino acid sequence DYKDDDDK (SEQ ID NO: 1); a peptide tag having the amino acid sequence CDYKDDDDK (SEQ ID NO: 8); a peptide tag having the amino acid sequence KRITVEEALAHPYLEQYYDPTDE (SEQ ID NO: 2; a peptide tag having the amino acid sequence HHHHHH (SEQ ID NO: 3); a peptide tag having the amino acid sequence EQKLISEEDL (SEQ ID NO: 4); a peptide tag having the amino acid sequence YPYDVPDYA (SEQ ID NO: 5); YTDIEMNRLGK (SEQ ID NO: 6); or a peptide tag having the amino acid sequence QPELAPEDPED (SEQ ID NO: 7). For example, the binding region of the anti-peptide agent may be highly selective for at least 60% of amino acid sequence of at least one of the peptide tags, such as 70%, 80%, 90%, 95%, or 100%, of the amino acid sequence of at least one of the peptide tags. Commercially available anti-peptide antibodies antibodies that are highly selective for a peptide tag having the amino acid sequence DYKDDDDK (SEQ ID NO: 1) include Sigma-Aldrich product codes F7425, F3040, F1804, F3165, F4042, F2555 and SAB4200071. In certain embodiments, the anti-peptide agent may be selective to a peptide tag only when the peptide tag occupies a certain type of position on a peptide-tagged agent, for example a peptide attached at the N-terminal position. In other embodiments, the anti-peptide agent may be insensitive to how a peptide tag is positioned on an anti-peptide agent.

In certain embodiments, a plurality of anti-peptide agents may be present at a ratio of at least 100:1, 125:1, 150:1, 200:1, 250:1, 300:1, 400:1, 500:1, 750:1, or 1000:1, on a weight:weight basis, relative to the plurality of peptide-tagged agents. In certain embodiments, a plurality of anti-peptide agents may be present at a ratio of in a range of 100:1-1000:1, 100:1-500:1, 100:1-300:1, 250:1-300:1, 300:1-1000:1, 300:1-500:1, 300:1-400:1, 300:1-350:1, 325:1-375:1, 300:1-310:1, 310:1-320:1, 320:1-330:1, 330:1-340:1, 340:1-350:1, or 350:1-360:1, on a weight:weight basis, relative to the plurality of peptide-tagged agents.

In certain embodiments, a plurality of anti-peptide agents may be present at a ratio of at least 100:1, 125:1, 150:1, 200:1, 250:1, 300:1, 400:1, 500:1, 750:1, or 1000:1, on a mole:mole basis, relative to the plurality of peptide-tagged agents. In certain embodiments, a plurality of anti-peptide agents may be present at a ratio of in a range of 100:1-1000:1, 100:1-500:1, 100:1-300:1, 250:1-300:1, 300:1-1000:1, 300:1-500:1, 300:1-400:1, 300:1-350:1, 325:1-375:1, 300:1-310:1, 310:1-320:1, 320:1-330:1, 330:1-340:1, 340:1-350:1, or 350:1-360:1, on a mole:mole basis, relative to the plurality of peptide-tagged agents.

A detectable agent may comprise, for example, an agent that may be capable of binding with the analyte, for example an antibody, inclusive, for example, of a monoclonal antibody, a polyclonal antibody, a multivalent antibody, a chimeric antibody, a multispecific antibody, or an antibody fragment; an aptamer; an affimer; a protein; a protein receptor; a protein ligand; or a fusion protein, comprising, for example, an immunoglobulin fusion partner, a fusion partner that stabilizes a receptor or a ligand, or a fusion partner that provides a target for binding, or a fusion partner that provides a detectable signal. In certain embodiments, the detectable agent may be capable of binding to an epitope of the analyte, for example the detectable agent may comprise a complementary determinant region, or a portion thereof, that may be capable of binding with one or more epitopes of the analyte, for example a phospho-epitope.

In certain embodiments, the detectable agent may have a molecular weight in a range of 100 Da to 10,000 kDa, for example a molecular weight in a range of 100 Da to 1 kDa, 1 kDa to 10 kDa, 10 kDa to 25 kDa, 25 kDa to 50 kDa, 50 kDa to 75 kDa, 75 kDa to 100 kDa, 100 kDa to 125 kDa, 125 kDa to 150 kDa, 150 kDa to 175 kDa, 175 kDa to 200 kDa, 200 kDa to 250 kDa, 250 kDa to 500 kDa, 500 kDa to 1,000 kDa, 1,000 kDa to 5,000 kDa, 5,000 kDa to 10,000 kDa, 75 kDa to 400 kDa, 100 kDa to 200 kDa, or a molecular weight in a range of 125 kDa to 175 kDa.

In certain embodiments, the analyte may be a target for the detectable agent. In certain embodiments, the detectable agent may be non-covalently bound to the analyte, for example releasably bound, partially bonded, hydrogen-bonded, ionically bonded, or a combination of two or more thereof.

In certain embodiments, one or more of the detectable agents may comprise, for example, a detectable tag that may be capable of producing a detectable signal. In certain embodiments, the magnitude, or another characteristic, of the signal may be related to the quantity or concentration of an analyte in a sample. In certain embodiments, the detectable tag may be applied to or bound to another portion of the detectable agent to form the complete detectable agent. In certain embodiments, the detectable tag may be integral to the rest of the detectable agent. For example, the detectable agent may include the detectable tag as a fusion partner, a labelled amino acid, or a labelled nucleotide.

Suitable detectable tags may include, for example, antigens; enzymes; fluorophores; quenchers; radioactive isotopes; luminescent labels; chemiluminescent labels; one or more lanthanide ions, for example Eu³⁺, Sm³⁺, Tb³⁺, and/or Dy³⁺, nucleic acids that may be capable of PCR amplification; coloured particles, inclusive, for example, of coloured latex beads, metal sols (for example, collidal gold), gold labels, or dye labels; tags that may be capable of interaction by fluorescence resonance energy transfer; and tags that may be capable of chemical transfer proximity interaction.

In certain embodiments, the detectable signal may be produced directly by the detectable agent or detectable tag, if present, or indirectly by a further molecule that can produce a detectable signal, such as, for example, a further molecule that may comprise an enzyme. In certain embodiments, detectable signals include, but are not limited to, light signals, inclusive of, for example, signals having a color or no color that may be detectable by human vision; stains; and signals that may be detected with a detector, inclusive of, for example, a spectrophotometer, a fluorometer, for example a fluorometer capable of measuring time-resolved fluorescence, a luminometer, a radioactivity-based counter, or an electrochemical detection apparatus. In certain embodiments, the detector may be a hand-held detector, a detector in a laboratory, and/or a mobile detector. In certain embodiments, detectable signals may include the lack of any of the foregoing types of signals, for example a quenched signal, when measured by a detector capable of detecting a signal if one were present.

Metal sols and other types of colored particles useful as detectable tags in immunoassay procedures are known. See, for example, U.S. Pat. No. 4,313,734; Horisberger, Evaluation of Colloidal Gold as a Cytochromic Marker for Transmission and Scanning Electron Microscopy, Biol. Cellulaire, 36, 253 258 (1979); Leuvering et al., Sol Particle Immunoassay, J. Immunoassay, 1 (1): 77 91 (1980), and Frens, Controlled Nucleation for the Regulation of the Particle Size in Monodisperse Gold Suspensions, Nature, Physical Science, 241: 20 22 (1973).

Use of metal sols, for example colloidal gold, may produce a visibly detectable signal. In certain embodiments, detectable tags may comprise, for example, colloidal gold providing a red detectable signal. In certain embodiments, detectable tags may comprise, for example, colloidal gold having a mean particle size in a range of 50-100 nm, such as, for example, colloidal gold having a mean particle size in a range of 55-90, 55-85, 40-47, 60-80, 60-75, or 65-75 nm. In certain embodiments, detectable tags may comprise, for example, colloidal gold having a mean particle size greater than 50 nm, greater than 52 nm, greater than 55 nm, greater than 57 nm, greater than 60 nm, or greater than 100 nm in size.

In certain embodiments, detectable tags may comprise, for example, particles, at least a portion of which may be substantially or predominantly spherical in shape. In certain embodiments, at least a portion of the detectable tags may include particles having substantially or predominantly non-spherical shape or shapes. In certain embodiments, detectable tags may comprise, for example, particles that may be substantially or predominantly monodisperse or have a narrow particle size distribution, for example a monodispersity greater than 50%, 75% or 95% as determined by a Coulter N4 Particle Analyzer (Beckman Coulter, Fullerton, Calif.).

Antigens that may be used as detectable tags may include, for example, any antigenic component of a detectable agent that may be targeted by a secondary detectable agent. In certain embodiments, an antibody may be used as a secondary detectable agent to detect an antigen detectable tag. In certain further embodiments, the antibody secondary detectable agent may be fluorescently or enzymatically labelled. In embodiments where the portion of the detectable agent that may bind with the analyte is part of an antibody, the antibody secondary detectable agent may have a binding affinity to an antigen on the detectable agent.

Enzymes that may be used as detectable tags include, for example, enzymes that result in the conversion of a portion of the detectable agent into a detectable product. In certain embodiments, the conversion may result in a change in colour or fluorescence or generation of an electrochemical signal by the detectable agent. Such enzymes may include, for example, horseradish peroxidase (HRP), alkaline phosphatase (AP), β-galactosidase, acetylcholinesterase, luciferase, or catalase.

Radioactive isotopes that may be used as detectable tags include, for example, ³H, ¹⁴C, ³²P, ³⁵S, or ¹³¹I. In certain embodiments, the radioisotope may be conjugated to a detectable agent or incorporated into a detectable agent by translation of mRNA encoding the detectable agent in the presence of radiolabelled amino acids. Radioisotopes and methods for conjugating radioactive isotopes to molecules such as proteins are known in the art and include methods discussed by Slater (Radioisotopes in Biology: A Practical Approach, Oxford University Press, 2002). Radioisotopes may be detected using gamma, beta or scintillation counters.

Fluorophores that may be used as detectable tags include, for example, resorufin; fluorescein, inclusive of, for example, fluorescein isothiocyanate, FITC; rhodamine, inclusive of, for example, tetramethyl rhodamine isothiocyanate, TRITC; green fluorescent protein, GFP; and phycobiliproteins, inclusive of, for example, allophycocyanin, phycocyanin, phycoerythrin and phycoerythrocyanin, or derivatives of any of the foregoing.

In certain embodiments, fluorophores may be subjected to applied stimulation, for example light of a suitable excitation wavelength to promote fluorescence. Alternatively, in certain embodiments stimulation may be provided by a fluorescence resonance energy transfer (FRET) partner, for example a donor molecule. When the fluorophore comes into close vicinity to the FRET partner, for example during formation of the analyte complex, the fluorophore may become excited by the FRET partner and fluoresce. FRET donors may include luminescent and/or fluorescent agents.

In certain embodiments, a detectable tag may comprise, for example, a quencher. Quenchers may be able to absorb excitation energy from fluorophores and may be used to suppress the fluorophore's emission when in close proximity. In this regard, the reaction is similar to a FRET reaction, except that the readout is a loss of fluorescence.

Luminescent compounds that may be used as detectable tags include, for example, chemiluminescent and bioluminescent compounds. In certain embodiments, these compounds may be used to label the detectable agent. In certain embodiments, the presence of the chemiluminescent-tag may be determined by detecting the presence of luminescence that arises during the course of a chemical reaction. Examples of useful chemiluminescent labelling compounds include, but are not limited to, luminol, isoluminol, theromatic acridinium ester, imidazole, acridinium salt and oxalate ester. In certain embodiments, the presence of a bioluminescent antibody is determined by detecting the presence of luminescence. Examples of bioluminescent compounds include, but are not limited to luciferin, luciferase and aequorin.

Nucleic acids that may be used as detectable tags include a suitable nucleic acid that may be capable of PCR amplification and/or hybridisation to a probe. The nucleic acid may be of sufficient length to allow binding of a forward and/or a reverse primer. In certain embodiments, the nucleic acid tag may be included in an aptamer or bound to a protein. In certain embodiments, nucleic acid detectable tags may be detected by performing a PCR reaction, whereby the nucleic acid tag is amplified and measured, or using a labelled nucleic acid probe with a complementary sequence to at least a portion of the nucleic acid tag. Methods of preparing and binding detectable nucleic acid tags to detectable agents are known in the art and include methods described in US 2009/0053701.

Detectable tags that may be capable of detection derived from chemical transfer proximity interaction may comprise, for example, a photoactive indicator precursor, for example a photoactive indicator precursor bound to a particle (such as a bead), which may be activated (for example, activated by singlet oxygen) to form a photoactive indicator. In certain embodiments, the photoactive indicator may be stimulated (for example, by irradiation) to produce a measurable light signal. Suitable photoactive indicator precursors may include, for example, rubrene, europium, europium chelate, samarium, or terbium. In embodiments where a photoactive indicator precursor is bound to a bead, suitable beads may include, for example, latex beads and magnetic beads.

In certain embodiments, a photoactive indicator may be capable of producing a light signal when irradiated by light. In certain further embodiments, the irradiating light may have a wavelength in a range of 250-1100 nm, for example, light at a wavelength in a range of 300-1000 nm, between 450-950 nm, between 360-441 nm, between 620-700 nm, between 600-630 nm, between 620-650 nm, between 640-700 nm, between 650-700 nm, between 670-690 nm, between 680-700 nm, or between 660-680 nm, such as by light at a wavelength of 620 nm, 630 nm, 640 nm, 650 nm, 660 nm, 670 nm, 675 nm, 680 nm, 685 nm, 690 nm, or at a wavelength of 700 nm.

In certain embodiments, the photoactive indicator, when irradiated, may fluoresce (i.e., emit a fluorescence) at a wavelength in a range of 500-625 nm, such as at a wavelength in a range of 525-575 nm, between 525-550 nm, between 540-560 nm, between 540-550 nm, 590-620 nm, between 600-625 nm, or in the range of 610-620 nm, such as at a wavelength of 520 nm, 530 nm, 535 nm, 540 nm, 545 nm, 550 nm, 555 nm, 560 nm, 600 nm, 605 nm, 610 nm, 615 nm, 620 nm, or at a wavelength of 625 nm.

In certain embodiments, detection of an analyte may comprise detecting a loss or gain of a light emission that may be derived from an interaction between at least a portion of the plurality of detectable tags and at least a portion of the permeable detection members. In certain embodiments, for example in certain embodiments utilizing a quencher, a FRET fluorophore, or photoactive indicator precursor as at least one of the detectable tags, an interacting component may be provided on, or integrated with, at least a portion of the permeable detection member. For example, in certain embodiments where at least a portion of the detectable tags comprise a fluorophore, the at least a portion of the permeable detection member may comprise a FRET partner (either donor or acceptor) or a quencher. In certain embodiments, in other embodiments where at least a portion of the plurality of detectable tags comprises a quencher, the at least a portion of the permeable detection member may comprise a suitable fluorophore. In certain embodiments, at least a portion of the plurality of detectable tags may comprise a chemical transfer interaction acceptor, for example a photoactive indicator precursor and, optionally, at least a portion of the permeable detection members may comprise a chemical transfer interaction donor, for example a donor capable of providing singlet oxygen upon stimulation by an appropriate light source. In certain embodiments, at least a portion of the plurality of permeable detection members may comprise a chemical transfer interaction acceptor, for example a photoactive indicator precursor and, optionally, at least a portion of the detectable tags may comprise a chemical transfer interaction donor, for example a donor capable of providing singlet oxygen upon stimulation by an appropriate light source.

In certain embodiments, detecting the presence of the immobilised analyte complex on at least a portion of the permeable detection membrane may utilise time-resolved fluorescence (TRF) and (fluorescence resonance energy transfer) FRET technologies, inclusive of, for example, the TRF-FRET technologies as described in EP 569,496, U.S. Pat. Nos. 5,527,684 or 6,861,264.

In certain embodiments, a plurality of anti-peptide agents may be present at a ratio of at least 0.1:1, 1:1, 2:1, 5:1, 8:1, 10:1, 15:1, 25:1, 50:1, 100:1, 300:1, or 500:1, on a weight:weight basis, relative to a plurality of detectable agents. In certain embodiments, a plurality of anti-peptide agents may be present at a ratio of in a range of 0.1:1-500:1, 0.1:1-300:1, 0.1:1-300:1, 0.1:1-500:1, 2:1-500:1, 2:1-100:1, 2:1-50:1, 2:1-25:1, 2:1-15:1, 5:1-15:1, 5:1-15:1, 5:1-10:1, 5:1-8:1, or 8:1-10:1, on a weight:weight basis, relative to the plurality of detectable agents.

In certain embodiments, a plurality of anti-peptide agents may be present at a ratio of at least 0.1:1, 1:1, 2:1, 5:1, 8:1, 10:1, 15:1, 25:1, 50:1, 100:1, 300:1, or 500:1, on a mole:mole basis, relative to a plurality of detectable agents. In certain embodiments, a plurality of anti-peptide agents may be present at a ratio of in a range of 0.1:1-500:1, 0.1:1-300:1, 0.1:1-300:1, 0.1:1-500:1, 2:1-500:1, 2:1-100:1, 2:1-50:1, 2:1-25:1, 2:1-15:1, 5:1-15:1, 5:1-15:1, 5:1-10:1, 5:1-8:1, or 8:1-10:1, on a mole:mole basis, relative to the plurality of detectable agents.

In certain embodiments, a plurality of detectable agents may be present at a ratio of at least 0.1:1, 1:1, 2:1, 5:1, 8:1, 10:1, 15:1, 25:1, 50:1, 100:1, 300:1, or 500:1, on a weight:weight basis, relative to a plurality of peptide-tagged agents. In certain further embodiments, the plurality of detectable agents may be present at a ratio of in a range of 0.1:1-500:1, 0.1:1-300:1, 0.1:1-300:1, 0.1:1-500:1, 2:1-500:1, 2:1-100:1, 2:1-50:1, 10:1-50:1, 20:1-50:1, 25:1-45:1, 30:1-45:1, 30:1-40:1, 35:1-40:1, or 37:1-40:1, on a weight:weight basis, relative to a plurality of peptide-tagged agents.

In certain embodiments, a plurality of detectable agents may be present at a ratio of at least 0.1:1, 1:1, 2:1, 5:1, 8:1, 10:1, 15:1, 25:1, 50:1, 100:1, 300:1, or 500:1, on a weight:weight basis, relative to a plurality of peptide-tagged agents. In certain further embodiments, the plurality of detectable agents may be present at a ratio of in a range of 0.1:1-500:1, 0.1:1-300:1, 0.1:1-300:1, 0.1:1-500:1, 2:1-500:1, 2:1-100:1, 2:1-50:1, 10:1-50:1, 20:1-50:1, 25:1-45:1, 30:1-45:1, 30:1-40:1, 35:1-40:1, or 37:1-40:1, on a weight:weight basis, relative to a plurality of peptide-tagged agents.

In certain embodiments, a plurality of anti-peptide agents may be present at a ratio in a range of 5:1-20:1, on a weight:weight basis, relative to the plurality of detectable agents, and the plurality of anti-peptide agents may be present at a ratio of in a range of 100:1-500:1, on a weight:weight basis, relative to a plurality of peptide-tagged agents. In certain embodiments, a plurality of anti-peptide agents may be present at a ratio in a range of 5:1-10:1, on a weight:weight basis, relative to the plurality of detectable agents and the plurality of anti-peptide agents may be present at a ratio of in a range of 150:1-400:1, on a weight:weight basis, relative to a plurality of peptide-tagged agents. In certain embodiments, a plurality of anti-peptide agents may be present at a ratio in a range of 5:1-10:1, on a weight:weight basis, relative to the plurality of detectable agents and the plurality of anti-peptide agents may be present at a ratio of in a range of 200:1-500:1, on a weight:weight basis, relative to a plurality of peptide-tagged agents. In certain embodiments, a plurality of anti-peptide agents may be present at a ratio in a range of 6:1-10:1, on a weight:weight basis, relative to the plurality of detectable agents and the plurality of anti-peptide agents may be present at a ratio of in a range of 250:1-400:1, on a weight:weight basis, relative to a plurality of peptide-tagged agents.

In certain embodiments, a plurality of anti-peptide agents may be present at a ratio in a range of 5:1-20:1, on a mole:mole basis, relative to the plurality of detectable agents, and the plurality of anti-peptide agents may be present at a ratio of in a range of 100:1-500:1, on a mole:mole basis, relative to a plurality of peptide-tagged agents. In certain embodiments, a plurality of anti-peptide agents may be present at a ratio in a range of 5:1-10:1, on a mole:mole basis, relative to the plurality of detectable agents, and the plurality of anti-peptide agents may be present at a ratio of in a range of 150:1-400:1, on a mole:mole basis, relative to a plurality of peptide-tagged agents. In certain embodiments, a plurality of anti-peptide agents may be present at a ratio in a range of 5:1-10:1, on a mole:mole basis, relative to the plurality of detectable agents, and the plurality of anti-peptide agents may be present at a ratio of in a range of 200:1-500:1, on a mole:mole basis, relative to a plurality of peptide-tagged agents. In certain embodiments, a plurality of anti-peptide agents may be present at a ratio in a range of 6:1-10:1, on a mole:mole basis, relative to the plurality of detectable agents, and the plurality of anti-peptide agents may be present at a ratio of in a range of 250:1-400:1, on a mole:mole basis, relative to a plurality of peptide-tagged agents.

In certain embodiments, the analyte complex may comprise, for example, the analyte bound to one peptide-tagged agent. In certain embodiments, the analyte complex may comprise, for example, the analyte bound to a plurality of peptide-tagged agents, for example the analyte bound to 2, 3, less than 5, less than 10, or bound to in the range of 10-20 peptide-tagged agents. In certain embodiments, the analyte complex may further comprise, for example, the analyte bound to one detectable agent. In other embodiments, the analyte complex may comprise, for example, the analyte bound to a plurality of detectable agents, for example the analyte bound to 2, 3, less than 5, less than 10, or bound to in the range of 10-20 detectable agents. For example, the analyte complex may comprise the analyte bound to 1 peptide-tagged agent and 1 detectable agent, or 2 peptide-tagged agents and 1 detectable agent. In certain embodiments, the analyte complex may comprise, for example, a peptide-tagged agent bound to 2 or more analytes, for example the peptide-tagged agent bound to 2, 3, less than 5, less than 10, or bound to in the range of 10-20 analytes. In certain embodiments, the analyte complex may comprise, for example, a detectable agent bound to 2 or more analytes, for example the detectable agent bound to 2, 3, less than 5, less than 10, or bound to in the range of 10-20 analytes. For example, the analyte complex may comprise 3 analytes bound to 2 peptide-tagged agents and 1, 2, or 3 detectable agents; or, for example, the analyte complex may comprise 2 analytes bound to 3 peptide-tagged agents and 6 detectable agent.

In certain embodiments, at least one peptide-tagged agent may be capable of binding to a first epitope of the analyte and at least one detectable agents may be capable of binding to a second epitope of the analyte. In certain embodiments, the type of epitope of the first epitope of the analyte and the second epitope of the analyte may be the same type of epitope. In certain embodiments, the type of epitope of the first epitope of the analyte and the second epitope of the analyte may be two different epitope types. In certain embodiments, the type of epitope of the first epitope of the analyte and the second epitope of the analyte may share portions of their sequence identify, for example at least 75%, 80%, 85%, 90%, or at least 95% shared sequence identity; or at least 75%, 80%, 85%, 90%, or at least 95% shared sequences with their sequences but not necessarily at the same positions relative to the terminal portions of the sequences. In certain embodiments, the at least one peptide-tagged agent and the at least one detectable agent may be the same type of antibody. In certain embodiments, the at least one peptide-tagged agent and the at least one detectable agent may be different types of antibodies. In certain embodiments, the first epitope of the analyte and/or the second epitope of the analyte is a phospho-epitope. In certain embodiments, the first epitope of the analyte and the second epitope of the analyte may overlap in a common portion of the analyte. In certain embodiments, the first epitope of the analyte and the second epitope of the analyte may not overlap but may be in close proximity to each other, for example within 50 nm of one another, for example within 25 nm, within 10 nm, within 5 nm, within 1 nm, or within 5 Angstroms of one another. In certain embodiments, the first epitope of the analyte and the second epitope of the analyte may be distal from one another, for example at least 1 nm apart from one another, for example at least 2 nm, 5 nm, 10 nm, 25 nm, or at least 50 nm apart from one another. In certain embodiments, the first epitope of the analyte and the second epitope of the analyte may be in the range of 10 Angstroms-10 nm of one another, for example 10 Angstroms-5 nm, 10 Angstroms-3 nm, or in the range of 25 Angstroms-3 nm of one another. In certain embodiments, the first epitope of the analyte and the second epitope of the analyte do not overlap and may be sufficiently distal to each such that no steric interactions or substantially no steric interactions occur between the at least one peptide-tagged agent and the at least one detectable agent. In certain embodiments, the first and second epitopes may be bound by the at least one peptide-tagged agent and the at least one detectable agent, respectively.

Certain embodiments may provide an assay platform for detecting an analyte. In certain embodiments, the assay platform may comprise, for example, a permeable zone that may be configured, for example, to transport at least a portion of a sample that may comprise the analyte, and optionally a further permeable zone which may be configured, for example, to receive at least a portion of the sample. In certain embodiments, the permeable zone may comprise, for example, a plurality of peptide-tagged agents. In certain embodiments, the plurality of peptide-tagged agents may be capable of forming an analyte complex that may comprise at least one of the plurality of peptide-tagged agents and the analyte. In certain embodiments, the further permeable zone may be configured to receive at least a portion of the portion of the sample from the permeable zone. In certain embodiments, the further permeable zone may comprise, for example, a plurality of anti-peptide agents present, for example, at a ratio of at least 100:1, one a weight:weight and/or mole:mole basis, relative to the plurality of peptide-tagged agents. In certain embodiments, the plurality of anti-peptide agents may be configured, for example, to immobilize the at least one of the plurality of peptide-tagged agents. In certain embodiments, the plurality of anti-peptide agents may be configured, for example, to immobilize the analyte complex (for example, the plurality of anti-peptide agents may be configured, for example, to immobilize the analyte complex with a probability of 0.01-99.95%, for example a probability of 10-99.95%, 10-25%, 10-50%, 25-75%, 50%-75%, or with a probability of 75-99.95%). In certain further embodiments, the plurality of anti-peptide agents may be configured, for example, to immobilize the at least 5% of the plurality of peptide-tagged agents, for example at least 10%, 20%, 30%, 40%, 50%, 60%, 70%, 80%, 90%, 95%, 99%, or at least 99.9% of the plurality of peptide-tagged agents, or all of the plurality of peptide-tagged agents. In certain embodiments, the plurality of anti-peptide agents may be configured, for example, to immobilize in the range of 5%-100% of the plurality of peptide-tagged agents, for example in the range of 10%-95%, 10%-75%, 10%-50%, 25%-95%, 25%-75%, 20%-50%, 50%-100%, 50%-75%, 75%-100%, or to immobilize in the range of 75%-99.95% of the plurality of peptide-tagged agents.

In certain embodiments, the permeable zone may be distinguishable from another portion of the assay platform, for example the further permeable zone. For example, in certain embodiments, distinguishable features of the permeable zone may be inclusive of, but not limited to, any one or more of the following features: comprising a plurality of peptide-tagged agents; configured to be the portion of the apparatus to which the sample is introduced; configured to be the portion of the apparatus to which the analyte is introduced; configured to bring the analyte into contact with the plurality of peptite-tagged agents and/or detectable agents; configured to communicate at least a portion of the sample (and/or a portion of the plurality of peptide-tagged agents, detectable agents, analyte, and/or analyte complex) by passive transport, capillary action, wetting, wicking, or a combination of two or more thereof; comprising a material that may be, for example, absorbent, fibrous, or porous; and comprising a release zone configured to introduce at least a portion of the peptide-tagged agents and/or detectable agents into the sample. Other distinguishing features of the permeable zone are contemplated herein. In certain further embodiments, the another portion, for example the further permeable zone, may not comprise one or more of the foregoing distinguishable features.

In certain embodiments, the further permeable zone may be distinguishable from another portion of the assay platform, for example the permeable zone. For example, in certain embodiments, distinguishable features of the further permeable zone may be inclusive of, but not limited to, any one or more of the following features: comprising a plurality of anti-peptide agents; configured to receive at least a portion of the sample (and/or a portion of the plurality of peptide-tagged agents, detectable agents, analyte, and/or analyte complex) by passive transport, capillary action, wetting, wicking, or a combination of two or more thereof; comprising a detection zone that may be configured to be the portion of the apparatus to which the analyte complex may be immobilized; configured to be the portion of the apparatus to which the analyte may be detected; comprising a material that may be, for example, absorbent, fibrous, or porous; and comprising a membrane, inclusive of, for example, a nitrocellulose membrane. Other distinguishing features of the further permeable zone are contemplated herein.

In certain embodiments, the permeable zone and the further permeable zone may be non-overlapping. In other embodiments, at least a portion of the permeable zone and at least a portion of the further permeable zone may overlap. In certain embodiments, the permeable zone may be contained within the further permeable zone. In certain embodiments, the further permeable zone may be contained within the permeable zone.

Certain embodiments may provide a lateral flow device. In certain embodiments, the lateral flow device may comprise, for example, a flow path that may be defined by, for example, at least a portion of a permeable sub-assembly of the lateral flow device. In certain embodiments, the lateral flow device may further comprise, for example, a release zone that may comprise, for example, a plurality of peptide-tagged agents. In certain embodiments, the lateral flow device may further comprise, for example, a detection zone that may comprise, for example, a plurality of anti-peptide agents that may be present, for example, in the flow path at a ratio of at least 100:1, on a weight and/or mole:mole basis, relative to the plurality of peptide-tagged agents. In certain embodiments, the release zone may be configured, for example, to release at least a portion of said plurality of peptide-tagged agents into the flow path. In certain embodiments, at least one of said plurality of anti-peptide agents may be a binding partner with at least one of said plurality of peptide-tagged agents.

In certain embodiments, the permeable sub-assembly may comprise one or more permeable members, for example, 1, 2, 3, 4, or 5 permeable members. Examples of suitable permeable members are inclusive of, for example, for example, a permeable strip, a fibrous pad, and a porous membrane. In certain embodiments, one or more of the permeable members may be capable of being wetted. In certain embodiments, one or more of the permeable members may comprise matrix of solid material, such as fibers or particles, interspersed with open spaces. In certain embodiments, the open spaces may comprise, for example, pores, channels, microchannels, spaces between fibers, cusps formed by adjacent and/or packed particles (for example, particles such as microspheres), and the like. Other types of open spaces are contemplated herein. In certain embodiments, a sample, for example a liquid, suspension, or solution, may be introduced to a portion of the permeable sub-assembly and transported through the open spaces of at least a further portion of the sub-assembly by passive transport, capillary action, wetting, wicking, or a combination of two or more thereof.

In certain embodiments, the flow path may be characterized by the flux, or migration, of a sample being transported through at least a portion of the permeable sub-assembly. In certain embodiments, the flow path may characterized by the general, average, or net direction of flow resulting from transport of the sample and/or portions thereof through the open spaces in at least a portion of the sub-assembly. In certain embodiments, the flow path may not be uniform in a cross-section of the permeable sub-assembly. For example, in certain embodiments, a sample may flow or migrate more quickly through larger open spaces and/or near a boundary of the permeable sub-assembly, for example near or at a surface, for example a top surface, or an edge of the permeable sub-assembly.

In certain embodiments, at least a portion of the flow path may be approximately parallel, linear, convergent, or divergent. In certain embodiments, at least a portion of the flow path may be a radial flow path.

In certain embodiments, the release zone may comprise a portion of the permeable sub-assembly. In certain further embodiments, the plurality of peptide-tagged agents may be dried on, bound, releasably bound, or unbound to at least a portion of the permeable sub-assembly. In embodiments in which at least a portion of the plurality of anti-peptide agents may be unbound to the sub-assembly, the plurality of peptide-tagged agents may be introduced to a portion of the sub-assembly with the sample, or introduced separately from the sample and become dissolved or suspended in the sample in the open spaces of the permeable sub-assembly. In certain embodiments in which the plurality of peptide-tagged agents is bound or releasably bound in the release zone, the release zone may be configured to further release at least a portion of said plurality of peptide-tagged agents into the flow path. In certain embodiments, at least a portion of the plurality of peptide-tagged agents may migrate, dissolve, disconnect, and/or diffuse from at least a portion of the permeable sub-assembly, for example the release zone. In certain embodiments, at least a portion of the plurality of peptide-tagged agents may migrate, dissolve, disconnect, and/or diffuse from at least a portion of the permeable sub-assembly after contact with the sample and/or a solvent.

In certain embodiments, said plurality of anti-peptide agents may be configured, for example, to immobilize at least a fraction of the at least a portion of said plurality of peptide-tagged agents configured to be released, for example released in the detection zone of the sub-assembly. In certain further embodiments, at least one of the at least a portion of said plurality of peptide-tagged agents configured to be released may be capable of binding with an analyte prior to being immobilized by said plurality of anti-peptide agents.

In certain embodiments, the detection zone may comprise a portion of the permeable sub-assembly. In certain further embodiments, the plurality of anti-peptide agents may be bound to at least a portion of the permeable sub-assembly. In certain embodiments, the plurality of anti-peptide agents may be passively bound. In certain embodiments, the plurality of anti-peptide agents may be bound by non-covalent interactions. Examples of suitable interactions include, for example, electrostatic interactions, hydrophilic interactions, hydrophobic interactions, ionic electrostatic interactions, van de Waals interactions, or combinations of two or more such interactions. In some embodiments, the anti-peptide agent may be actively bound, for example covalently bound, to the solid substrate.

In certain embodiments, at least a portion of the permeable sub-assembly may comprise a plurality of linkers which may facilitate covalent bonding of the plurality of anti-peptide agents. Suitable linkers may include, for example, glutathione, maleic anhydride, a metal chelate, or meleimide. In certain further embodiments, the plurality of anti-peptide agents may be exposed to at least a portion of the open spaces in at least a portion of the permeable sub-assembly, for example the open spaces present in the detection zone.

In certain further embodiments, at least a portion of the plurality of detectable agents may be dried on, bound, releasably bound, or unbound to at least a portion the permeable sub-assembly. In certain embodiments in which at least a portion of the plurality of detectable agents may be unbound to the sub-assembly, the detectable agent may be introduced to a portion of the sub-assembly with the sample, or introduced separately from the sample and become dissolved or suspended in the sample on the permeable sub-assembly. In certain embodiments in which the plurality of detectable agents is bound or releasably bound in the release zone, the release zone may be configured to further release at least a portion of said plurality of detectable agents into the flow path. In certain further embodiments, at least a portion of the plurality of detectable agents may be capable of migrating, dissolving, disconnecting, or diffusing from at least a portion of the permeable sub-assembly, for example the release zone. In certain embodiments, at least a portion of the plurality of detectable agents may migrate, dissolve, disconnect, and/or diffuse from at least a portion of the permeable sub-assembly after contact with the sample and/or a solvent.

Certain embodiments may provide an assembly. In certain embodiments, the assembly may comprise, for example, a permeable release member that may have a plurality of peptide-tagged agents deposited on at least a portion thereof and, optionally, a plurality of detectable agents deposited on at least a portion thereof. In certain embodiments, the assembly may further comprise, for example, a permeable detection member that may be in fluid communication with the permeable release member. In certain embodiments, the permeable detection member may have a plurality of anti-peptide agents bound to at least a portion thereof.

In certain embodiments, the permeable release member may absorb at least a portion of the plurality of peptide-tagged agents and/or at least a portion of the plurality of detectable agents. In certain embodiments, the permeable release member may allow release of at least a portion of the plurality of peptide-tagged agents and/or at least a portion of the plurality of detectable agents. In certain embodiments, the permeable release member may provide structural support for at least a portion of the assembly. In certain embodiments, at least a portion of the plurality of peptide-tagged agents and/or at least a portion of the plurality of detectable agents may be striped onto the permeable release member. In certain embodiments, at least a portion of the plurality of peptide-tagged agents and/or at least a portion of the plurality of detectable agents may be dried onto the permeable release member. As explained herein, the striped and/or dried peptide-tagged agents and/or detectable agents may migrate, dissolve, disconnect, and/or diffuse from at least a portion of the permeable release member into a sample or a solvent. In certain embodiments, at least a portion of the plurality of peptide-tagged agents and/or plurality of detectable agents may be able to passively mix with a liquid sample present on the permeable release member. In certain embodiments, any one or more of the types of analyte complexes contemplated herein may be able to be formed on the permeable release pad.

In certain embodiments, the permeable release member may comprise a bibulous, hydrophilic material, such as, for example, an absorbent material. Suitable permeable release member materials may comprise, for example, cotton linter; cellulosic materials, or materials made of cellulose together with a polymeric fibrous material, such as polyamide or rayon fibers; and glass fiber material. For example, suitable materials may include cotton linter paper, such as S&S 903 and S&S GB002 (available from Schleicher and Schuell, Inc., Keene, N.H.), and BFC 180 (available from Whatman, Fairfield, N.J.); cellulosic materials, such as Grade 939 made of cellulose with polyamide, Grade 989 made of cellulose blend fiber, and Grade 1278 and Grade 1281 made of cellulose and rayon with polyamide (available from Ahlstrom Corporation, Mt. Holly Springs, Pa.); and glass fiber, such as Lydall borosilicate (available from Lydall, Inc., Rochester, N.H.).

In certain embodiments, the permeable release member may be blocked to prevent non-specific binding. In certain embodiments, the permeable release member may be coated with an aqueous solution containing bovine serum albumin (BSA) and a nonionic surfactant, such as polyethylene glycol, for example the polyethylene glycol sold under the trade name Triton X-100 (available from Rohm & Haas Co., Philadelphia, Pa.), for example, a combination of about 3% BSA and about 0.1% Triton X-100.

In certain embodiments, the permeable detection member may be formed of a substance which may permit binding of the plurality of anti-peptide agents thereto. In certain embodiments, the permeable detection member may comprise a polymeric material, for example a microporous film or membrane which may permit at least a portion of the anti-peptide agents to be passively bound or absorbed thereon. In such embodiments, it may not be necessary to bind the plurality of anti-peptide agents to the permeable detection member by chemical or physical fixation. In other embodiments, the plurality of anti-peptide agents may be bound to the permeable detection member by chemical or physical fixation.

In certain embodiments, suitable permeable detection member materials may comprise, for example, a microporous polymeric film of nitrocellulose, nylon, charge-modified nylon, polyvinylidine fluoride, polyethersulfone, or similar materials, or combinations of such materials. In certain embodiments, suitable permeable detection member materials may have a pore size in a range of between 0.1 μm and 20 μm, for example in a range of between 1 μm and 3 μm, 3 μm and 10 μm, 10 μm and 20 μm, or in a range of between 5 μm and 20 μm.

In certain embodiments, the permeable detection member may comprise a nitrocellulose membrane, inclusive of, for example, a nitrocellulose membrane having added detergents and/or surfactants. In certain embodiments, the permeable detection member may comprise a nitrocellulose membrane, inclusive of, for example, a membrane comprising nitrocellulose alone or a mixed ester of nitrocellulose, such as in combination with an ester of nitric acid and/or other acids. In certain further embodiments, a nitrocellulose membrane permeable detection member may be coated or laminated onto a translucent or transparent polymeric film to provide physical support for the membrane. In certain embodiments, the permeable detection member may comprise a nitrocellulose polymer which has been cast onto a polyester film, such as MYLAR®. In certain embodiments, the permeable detection member may comprise a nitrocellulose membrane laminated onto a polyester film, or laminated onto another backing materials besides polyester. Suitable backing materials may include, for example, pre-laminated or pre-cast planar sheets.

In certain embodiments, the permeable release member and the permeable detection member may be joined by overlapping a downstream edge of the permeable release member over an upstream edge of the permeable detection member, then adhering the resulting biphasic material to a clear polymer film or sheet, thereby holding the two members in place. In other embodiments, the permeable release member and the permeable detection member may be joined at a non-overlapping butt-joint, and adhered to a clear polymer film or sheet, thereby holding the two members in place. In certain embodiments, the joined members may provide a continuous flow path for transport of the sample, peptide-tagged agent, detectable agent, analyte, analyte complex, or any combination of the foregoing. Transport may comprise, for example, passive transport, capillary action, wetting, wicking, or a combination of two or more thereof. In certain embodiments, the joined members are in fluid communication.

In certain embodiments, at least one of the permeable release member and the permeable detection member has at least one flow channel spanning at least a portion of the respective member with a smallest cross-sectional width in a range of 3-20 times a diameter of a sphere having a molecular volume of the analyte complex. In certain embodiments, the at least one flow channel has a smallest cross-section width of less than 10, from 10 to 20, or greater than 20 times a diameter of a sphere having a molecular volume of the analyte complex.

A schematic view of a representative lateral flow detection apparatus 100 is illustrated in FIG. 1. According to this embodiment, absorbent sample pad 106, permeable release member 108, permeable detection member 110, and absorbent wick pad 112 are affixed onto backing card 102 into fluid communication. A liquid sample containing an analyte may be introduced to absorbent sample pad 106 and fluidly communicated by capillary action to permeable release member 108 where the analyte may encounter at least one peptide-tagged agent and, optionally, a detectable agent in the liquid sample, at which time it may bind to form an analyte complex. The analyte complex may then be fluidly communicated through the remainder of permeable release member 108 and be introduced to permeable detection member 110. The analyte complex will continue to flow in permeable detection member 110 unless and until it is immobilized thereon. A portion of the liquid sample, including potentially non-immobilized analyte complex, will flow through permeable detection member 110 and be absorbed into wick pad 112.

A schematic view of solvent-driven lateral flow detection apparatus 200 is illustrated in FIG. 2. According to this embodiment, absorbent solvent pad 204, permeable sample pad 206, permeable release member 208, permeable detection member 210, and absorbent wick pad 212 are affixed onto backing card 202 into fluid communication. A liquid solvent may be introduced to absorbent solvent pad 204 and fluidly communicated by capillary action to permeable sample pad 206 where the solvent may dissolve or suspend an analyte present in the sample. The analyte-containing solvent may then flow to permeable release member 208 where the analyte may encounter at least one peptide-tagged agent and, optionally, a detectable agent in the solvent, at which time the analyte may bind to form an analyte complex. The analyte complex may then be fluidly communicated through the remainder of permeable release member 208 and be introduced to permeable detection member 210. The analyte complex will continue to flow in permeable detection member 210 unless and until it is immobilized thereon. A portion of the solvent, including potentially non-immobilized analyte complex, will flow through permeable detection member 210 and be absorbed into wick pad 212.

A schematic view of multi-purpose lateral flow test kit 300 configurable to detect any one of three types of analytes is illustrated in FIG. 3. According to this embodiment, absorbent sample pad 306, universal receiver for a permeable release member 308, permeable detection member 310, and absorbent wick pad 312 are affixed onto backing card 302, bringing permeable detection member 310, and absorbent wick pad 312 into fluid communication. First permeable release member 308A, second permeable release member 308B, or third permeable release member 308C, each optionally containing a different type of peptide-tagged agent and/or detectable agent, may be inserted into universal receiver 308 to bring the inserted member into fluid communication with absorbent sample pad 306 and permeable detection member 310.

A schematic view of representative radial flow detection apparatus 400 for the detection of eight (8) different analytes is illustrated in FIG. 4. According to this embodiment, absorbent sample pad 406 is centrally positioned and in fluid communication with permeable release member 408, permeable detection member 410, and absorbent wick pad 412. A liquid sample optionally containing a plurality of different types of analytes may be introduced to absorbent sample pad 406 and flow radially (divergently) by capillary action to permeable release member 408 where each of the analytes may encounter at least one peptide-tagged agent to which it can bind and a detectable agent to which it can bind in the liquid sample, at which time it may bind to form an analyte complex. The analyte complex may then be fluidly communicated through the remainder of permeable release member 408 and be introduced to permeable detection member 410. The analyte complex will continue to flow in permeable detection member 410 unless and until it is immobilized thereon. A portion of the liquid sample, including potentially non-immobilized analyte complex, will flow through permeable detection member 410 and be absorbed into wick pad 412. In order to detectably distinguish between each of the up to eight types of analytes that may be present in the sample, apparatus 400 may be divided into eight sections, as delineated by dashed lines 414A-H. Within each section, a different plurality of peptide-tagged agents and a different plurality of detectable agents may be striped onto the portion of release zone 408 present in each section, forming stripes 408A-H. Within each section, the different plurality of peptide-tagged agents and the different plurality of detectable agents may be striped together, in sequence, or side by side relative to the direction of flow of the sample. Within each section, a plurality of anti-peptide agents may be striped onto the portion of detection zone 410 present in each section, forming stripes 410A-H. In certain exemplary embodiments, the type of anti-peptide agent present in each section may be the same. In other exemplary embodiments, different types of anti-peptide agents may be used in two or more of the sections. In certain embodiments, one or more of the sections may be in fluid communication. In certain embodiments, one or more of the sections may not be in fluid communication, for example one or more of the section delimiters indicated by dashed lines 414A-H may also denote the presence of a fluid barrier (e.g., a plastic wall) which prevents fluid communication between two or more sections. As is apparent from FIG. 4, in certain embodiments the radial flow detection apparatus may be readily configured to have fewer or more than eight (8) sections, and may thereby be configured to detect fewer or more than eight (8) different types of analytes.

A schematic view of a representative multi-analyte lateral flow detection apparatus 500 for the detection of six (6) different analytes is illustrated in FIG. 5. According to this embodiment, absorbent sample pad 506, permeable release member 508, permeable detection member 510, and absorbent wick pad 512 are affixed onto backing card 502 into fluid communication. A liquid sample optionally containing a plurality of different types of analyte may be introduced to absorbent sample pad 506 and fluidly communicated by capillary action to permeable release member 508 where each of the analytes may encounter at least one peptide-tagged agent to which it may bind and a detectable agent to which it may bind in the liquid sample, at which time it may bind to form an analyte complex. Each of the analyte complexes formed thereby may then be fluidly communicated through the remainder of permeable release member 508 and be introduced to permeable detection member 510. Each analyte complex will continue to flow in permeable detection member 510 unless and until it is immobilized thereon. A portion of the liquid sample, including potentially non-immobilized analyte complex, will flow through permeable detection member 510 and be absorbed into wick pad 512. Permeable release member 508 is striped with a plurality of six (6) types of peptide-tagged agents and with six (6) types of detectable agents in order to provide the types of agents required to bind with each of the six (6) types of analytes. The plurality of six (6) types of peptide-tagged agents and the plurality of six (6) types of detectable agents may be striped together in zone 508A. Alternatively, the plurality of six (6) types of peptide-tagged agents may be striped in zone 508A and the plurality of six (6) types of detectable agents may be striped together in zone 508B. Permeable detection member 510 may be striped with a plurality of up to six (6) or more types of anti-peptide agents, each capable of immobilization at least one of the types of peptide-tagged agents. The plurality of six (6) or more types of anti-peptide agents may be striped together in zone 510A. Alternatively, one or more additional zones 510B-F may be used to separately strip one or more of the plurality of six (6) or more types of anti-peptide agents. Additionally, permeable detection member 510 may comprise control zone 510G configured to provide an indication that the apparatus has functioned, for example by detectably capturing any type of particle present in the sample. As is apparent from FIG. 5, in certain embodiments the multi-analyte lateral flow detection apparatus may be readily configured to detect fewer or more than six (6) analytes by adjusting the number and types of peptide-tagged agents, detectable agents, and anti-peptide agents and the striping scheme.

### EXAMPLES

### Lateral Flow Test Strip Preparation

Release Pads:

Millipore GFDX glass fiber pads were blocked with blocking buffer (10 mM borate, 3% BSA, 1% PVP-40, 0.25% Tx-100), dried, and stored desiccated. The dried glass fiber pads were then sprayed with detectable agent and, optionally, a peptide-tagged agent, in accordance with Table 1 and then dried at approximately 40° C. and stored desiccated.

Detection Membranes:

Sartorius Unisart CN140 nitrocellulose membranes were striped with an immobilization agent in accordance with Table 1 using a Biodot frontline dispenser at a dispense rate of about 1 μL/cm, dried, and stored dessicated. The nitrocellulose membranes were then blocked with blocking buffer (10 mM phosphate, 0.1% sucrose, 0.1% BSA, 0.2% PVP-40, pH 7.2), dried at approximately 40° C., and stored desiccated.

TABLE 1
Conjugate Pad and Detection Membrane Preparations

| | Conjugate Pad | | Detection Membrane |
|---|---|---|---|
| Preparation | Peptide-Tagged Agent | Detectable Agent | Immobilization Agent |
| A | 6.5 ng anti-hCG-β tagged with | 250 ng anti-hCG-α | 2000 ng mouse monoclonal |
| | a plurality of peptide tags | conjugated to colloidal | IgG1 immobilization agent |
| | (sequence: CDYKDDDDK | gold | (capable of immobilizing the |
| | (SEQ ID NO: 8)) | | tagged anti-hCG- β) |
| B | (no Capture Agent was | 500 ng anti-hCG-α | 500 ng anti-hCG-β |
| | placed Conjugate Pad) | conjugated to colloidal | (capable of directly |
| | | gold | immobilizing hCG) |

Lamination and Assembly:

Each lateral flow test strip comprised: (1) a conjugate pad having a 2 mm overlap with the top of one end of a detection membrane, (2) a wick pad having a 2 mm overlap with the top of the opposite end of the detection membrane, and (3) an adhesive backing card onto which the conjugate pad—detection membrane—wick pad configuration was affixed. 160 test strips were used (80 each of based on Preparation A and Preparation B, respectively) in the following Example.

### Example 1

A series of hCG detection experiments were conducted at the concentrations shown in Table 2. In each experiment, 40 μL of hCG solution was deposited on the conjugate pad and allowed to run for 8 minutes. Afterwards, a wash solution was deposited on the conjugate pad and allowed to run for 7 minutes. Results were obtained by detection of detectable agent present in the test zone with an ESE TS012 Gold Reader and reported in Table 2.

TABLE 2
Detection Results for Example 1

| | | Lateral Flow Test | | | Lateral Flow Test | | |
|---|---|---|---|---|---|---|---|
| | | Strip Based On | | | Strip Based On | | |
| | | Preparation A | | | Preparation B | | |
| Experiment | Concentration | Average | Standard | Signal/ | Average | Standard | Signal/ |
| No. | hCG, mIU/mL | Signal* | Deviation | Noise | Signal* | Deviation | Noise |
| 1 | 0 | 8.3 | 4.96 | 1.00 | 16.4 | 9.99 | 1.00 |
| 2 | 0.25 | 13.9 | 5.82 | 1.67 | 14.2 | 7.51 | 0.87 |
| 3 | 0.5 | 16.5 | 6.41 | 1.99 | 20.0 | 5.16 | 1.22 |
| 4 | 1 | 16.2 | 6.71 | 1.95 | 13.0 | 6.28 | 0.80 |
| 5 | 2 | 33.3 | 12.60 | 4.01 | 19.3 | 6.75 | 1.18 |
| 6 | 5 | 71.9 | 17.45 | 8.66 | 49.4 | 7.59 | 3.02 |
| 7 | 10 | 112.4 | 15.01 | 13.54 | 90.0 | 7.22 | 5.51 |
| 8 | 15 | 164.3 | 20.42 | 19.80 | 121.5 | 14.89 | 7.43 |
| 9 | 25 | 197.7 | 32.45 | 23.82 | 177.3 | 24.34 | 10.84 |
| 10 | 50 | 349.8 | 35.40 | 42.14 | 279.5 | 11.87 | 17.10 |
| 11 | 100 | 451.5 | 65.08 | 54.39 | 389.1 | 20.75 | 23.80 |

*Each experiment was repeated 4-8 times, resulting in a total of 80 experiments each for Lateral Flow Test Strip Preparation A and Preparation B, respectively.

All publications and patent applications mentioned in this specification are herein incorporated by reference to the same extent as if each individual publication or patent application was specifically and individually indicated to be incorporated by reference.

While certain embodiments of the present invention have been shown and described herein, it will be obvious to those skilled in the art that such embodiments are provided by way of example only. It is intended that the following claims define the scope of the invention and that methods and structures within the scope of these claims and their equivalents be covered thereby.

1-20. (canceled)

21. A method to detect a predetermined analyte, comprising:
i) providing:
a) a first permeable release member comprising a plurality of first peptide-tagged agents and a plurality of detectable agents;
b) a universal detection member in fluid communication with the first permeable release member, the universal detection member comprising a porous membrane and a plurality of anti-peptide agents bound to the porous membrane, the plurality of anti-peptide agents present at a ratio of at least 200:1 on a weight:weight basis, relative to the plurality of first peptide-tagged agents, the plurality of first peptide-tagged agents having a plurality of covalently conjugated peptide tags; and
c) an absorbent wicking pad in fluid communication with the universal detection member;
ii) contacting the first permeable release member with a sample, the sample comprising the predetermined analyte;
iii) forming an analyte complex comprising the predetermined analyte bound to at least one of the plurality of first peptide-tagged agents and separately bound to at least one of the plurality of detectable agents;
iv) passively transporting the analyte complex by capillary flow action to the universal detection member;
v) immobilizing the analyte complex to the universal detection member by selectively binding at least one of the plurality of covalently conjugated peptide tags with at least one of the plurality of anti-peptide agents; and
vi) detecting the immobilized analyte complex by detecting the at least one of the plurality of detectable agents.

22. The method of claim 21, wherein the first permeable release member is selected from a plurality of permeable release members that are compatible for use with the universal detection member for detecting different types of predetermined analytes.

23. The method of claim 22, wherein the plurality of permeable release members comprises at least a second permeable release member, the at least a second permeable release member comprising a plurality of at least second peptide-tagged agents, at least one of the plurality of at least second peptide-tagged agents a specific binding partner with at least one of the plurality of anti-peptide agents.

24. The method of claim 21, further comprising: providing a universal receiver, the universal receiver configured to (a) receive the first permeable release member, and to (b) fixedly position the first permeable release member in fluid communication with the universal detection member.

25. The method of claim 24, wherein the universal receiver is configured to receive any one of a plurality of permeable release members for detecting different types of predetermined analytes.

26. The method of claim 21, wherein the plurality of anti-peptide agents is present at a ratio of at least 300:1, on a weight:weight basis, relative to the plurality of first peptide-tagged agents.

27. The method of claim 21, wherein the plurality of detectable agents and the plurality of first peptide-tagged agents are mixed and striped onto the first permeable release member.

28. The method of claim 21, wherein the plurality of detectable agents and the plurality of first peptide-tagged agents are separately striped onto the first permeable release member.

29. The method of claim 21, wherein the plurality of anti-peptide agents are present at a ratio of at least 8:1, on a weight:weight basis, relative to the plurality of detectable agents.

30. The method of claim 21, wherein the plurality of detectable agents are present at a ratio of at least 2:75, on a weight:weight basis, relative to the plurality of first peptide-tagged agents.

31. The method of claim 21, wherein at least one of the plurality of anti-peptide agents comprises a specific binding partner to at least one of the plurality of covalently conjugated peptide tags.

32. The method of claim 31, wherein at least one of the plurality of covalently conjugated peptide tags has the sequence CDYKDDDDK (SEQ ID NO: 8).

33. The method of claim 21, wherein the sample is formed by contacting a solvent with a material present on an absorbent sample pad, the absorbent sample pad in fluid communication with the first permeable release member.

34. The method of claim 21, wherein the predetermined analyte is detected within 10 minutes of the contacting.

35. A method to detect a predetermined analyte, comprising:
i) providing:
a) a first permeable release member comprising a capture agent and a detectable agent, the capture agent comprising a first antibody covalently conjugated to a plurality of first peptide tags, the detectable agent comprising a detectable tag;
b) a universal detection member in fluid communication with the permeable release member, the universal detection member comprising a porous membrane and an anti-peptide antibody bound to the porous membrane, wherein the anti-peptide antibody is a specific binding partner to at least one of the plurality of first peptide tags; and
c) an absorbent wicking pad in fluid communication with the universal detection member;
ii) contacting the first permeable release member with a sample, the sample comprising the predetermined analyte;
iii) forming an analyte complex comprising the predetermined analyte bound to the capture agent and separately bound to the detectable agent;
iv) passively transporting the analyte complex by capillary flow action to the universal detection member;
v) immobilizing the analyte complex to the universal detection member by selectively binding the anti-peptide antibody with at least one of the plurality of first peptide tags; and
vi) detecting the analyte within 10 minutes of the contacting by detecting the detectable tag,
wherein the first permeable release member is selected from a plurality of permeable release members that are compatible for use with the universal detection member for detecting different types of predetermined analytes, wherein the plurality of permeable release members comprises at least a second permeable release member, the at least a second permeable release member comprising at least a second antibody covalently conjugated to a plurality of second peptide tags, at least one of the plurality of second peptide tags a specific binding partner with the anti-peptide antibody.

36. A method for preparing a series of lateral flow devices, comprising:
i) preparing a series of porous membranes comprising a quantity of antibodies, the antibodies configured to selectively bind with a certain type of peptide;
ii) further preparing a series of absorbent pads comprising a quantity of antibody capture agents covalently conjugated to at least one of the certain type of peptide; and
iii) assembling the series of lateral flow devices, the series of lateral flow devices comprising:
a) at least one porous membrane from the series of porous membranes; and
b) at least one absorbent pad from the series of absorbent pads.

37. The method of claim 36, wherein the series of lateral flow devices comprises:
a) a first lateral flow device comprising a first porous membrane of the series of porous membranes and a first absorbent pad of the series of absorbent pads, the first absorbent pad comprising a quantity of first antibody capture agents; and
b) at least a second lateral flow device comprising at least a second porous membrane of the series of porous membranes and at least a second absorbent pad of the series of absorbent pads, the at least a second absorbent pad comprising a quantity of at least second antibody capture agents.

38. The method of claim 37, wherein the quantity of first antibody capture agents comprises a first type of antibody and the quantity of at least second antibody capture agents comprises a second type of, antibody, wherein the first type of antibody is different from the second type of antibody.
